(19) United States
(12) Patent Application Publication
(10) Pub. No.: US 2004/0229960 A1
(43) Pub. Date: Nov. 18, 2004

(54) Compositions and methods of administering tubulin binding agents for the treatment of ocular diseases

(76) Inventors: David Sherris, Jamaica Plains, MA (US); Mark Wood, Milton, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/732,680

(22) Filed: Dec. 9, 2003

Related U.S. Application Data
(63) Continuation-in-part of application No. 10/344,886, filed on Feb. 11, 2003.
Application No. 10/344,886 is a 371 of international application No. PCT/US02/22449, filed on Jul. 15, 2002.
(60) Provisional application No. 60/386,227, filed on Jul. 13, 2001.

(51) Int. Cl. A61K 31/075

(52) U.S. Cl. 514/720

(57) ABSTRACT

The present invention is directed to the administration of vascular targeting agents, particularly a tubulin binding agent, for the treatment of ocular neovascularization, ocular tumors, and conditions such as diabetic retinopathy, retinopathy of prematurity, retinoblastoma and macular degeneration.

### RELATED APPLICATIONS

This application is a continuation-in-part application of U.S. Ser. No. 10/344,886, which was filed on Jul. 15, 2002 and is a national phase case of US02/22449, which was filed on Jul. 15, 2002, which claims priority to U.S. Serial No. 60/386,227, which was filed on Jul. 13, 2001.

### FIELD OF THE INVENTION

The invention relates to the administration of vascular targeting agents, particularly tubulin binding agents, for the treatment of ocular diseases.

### BACKGROUND OF THE INVENTION

The eye is fundamentally one of the most important organs during life. Because of aging, diseases and other factors which can adversely affect vision, the ability to maintain the health of the eye becomes all important. A leading cause of blindness is the inability to introduce drugs or therapeutic agents into the eye and to maintain these drugs or agents at a therapeutically effective concentration therein. Oral ingestion of a drug or injection of a drug at a site other than the eye provides the drug systemically. However, such systemic administration does not provide effective levels of the drug specifically to the eye and thus may necessitate administration of often unacceptably high levels of the agent in order to achieve effective intraocular concentrations.

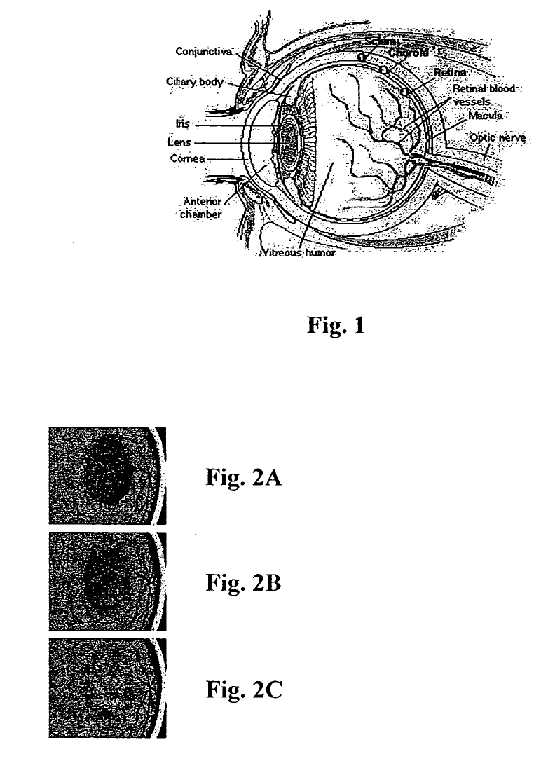

The macula is a region of the retina that contains an elevated concentration of the photo-sensor cells that are responsible for fine-detail vision (a generalized anatomic diagram of the human eye is illustrated in FIG. 1). Macular degeneration is the imprecise historical name given to a poorly understood group of diseases that cause the photo-sensor cells of the macula to lose function. The result of macular degeneration is the loss of vital central vision and detailed vision. A patient stricken with macular degeneration experiences a blank spot in the center of their visual field and often loses the ability to read small print. (Source: Macular Degeneration Foundation, San Jose, Calif.: www.eyesight.org)

Over 12 million Americans have some form of macular degeneration. One in six Americans between the ages of 55 and 64 will be affected by macular degeneration and the incidence of the disease increases with age. Each year 1.2 million of the estimated 12 million people with macular degeneration will suffer severe central vision loss. Each year 200,000 individuals will lose all central vision in one or both eyes.

Although the exact cause of macular degeneration is unknown, the architecture of the macula reveals clues as to how the disease might be initiated. The macula contains highly active photoreceptors that consume a great deal of energy. Generating this energy requires a rich supply of oxygen and nutrients. The macula has one of the highest rates of blood-flow through its supply-vessels (a.k.a. choroid). Anything that interferes with this rich blood supply can cause the macula to malfunction. The oxygen-deprived macula responds by producing cytokines that signal endothelial cell growth and neovascularization.

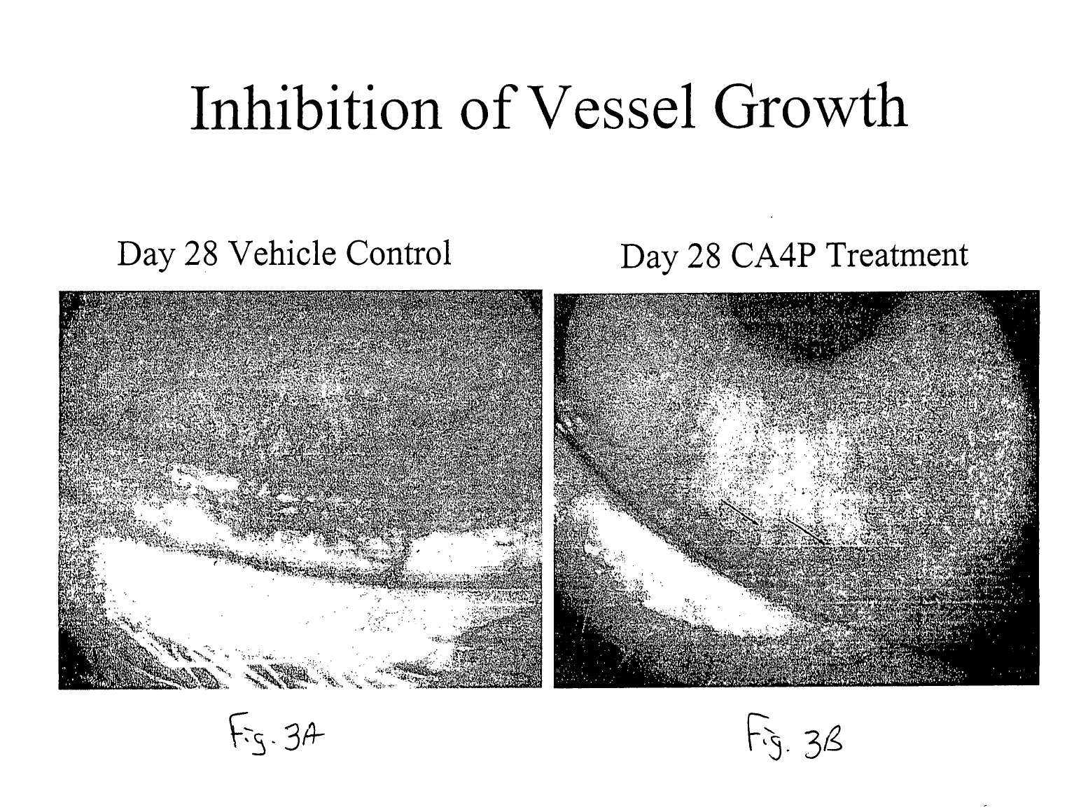

There are two basic types of macular degeneration: dry-form and wet-form. Approximately 85% to 90% of the cases of macular degeneration are the dry type. In the dry-form of the disease, the deterioration of the retina is associated with the formation of yellow deposits under the macula known as drusen. The deposition of drusen correlates with decrease in the thickness of retinal cells that comprise the macula. The amount of central vision loss is directly related to the location and severity of the drusen-induced retinal thinning. The dry-form of macular degeneration tends to progress more slowly than the wet-form of the disease. There is no effective treatment for dry-form macular degeneration. A small percentage individuals suffering from the dry-form of macular degeneration progress to the wet-form of macular degeneration. FIG. 2 illustrates a normal macula and dry-form macular degeneration.

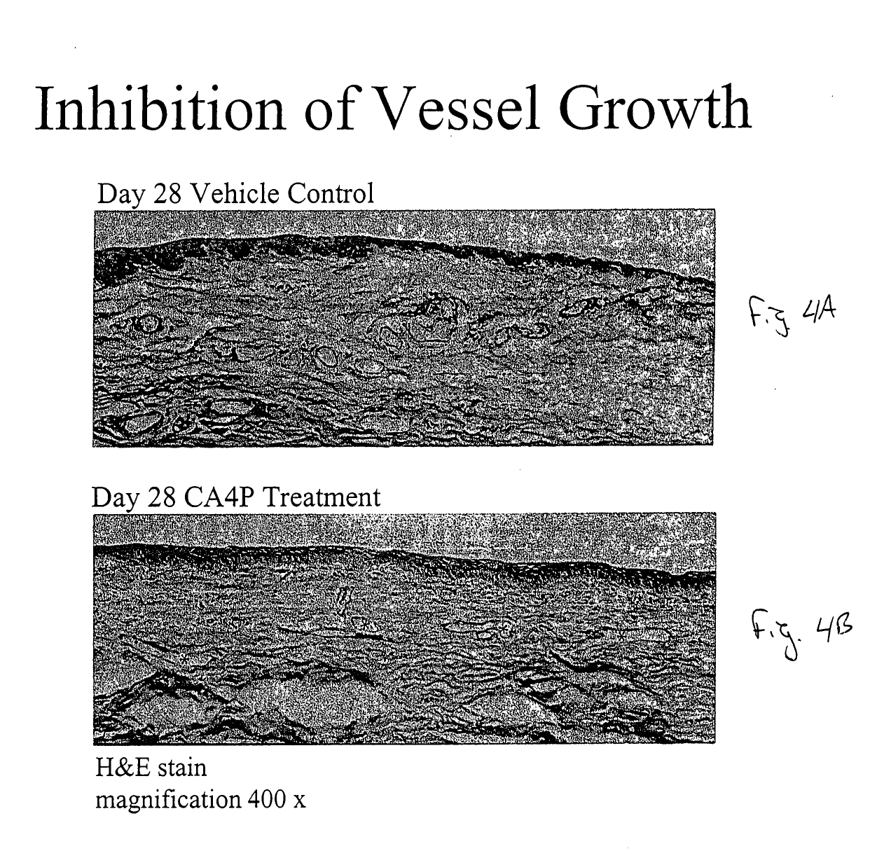

The wet-form of macular degeneration is a rapidly progressing disease that almost always results in severe vision loss. Vision-loss associated with Wet macular degeneration is the result of sub-retinal neovascularization. The rapid growth of the sub-retinal blood vessels causes the overlying layer of retinal cells to buckle and become detached from the nutrient-rich choroid. In extreme cases of Wet macular degeneration the proliferating vessels penetrate the retina and infiltrate the vitreous humor. Several treatments exist for wet-form neovascularization however none are remotely satisfactory. FIG. 3 illustrates a normal macula and wet-form macular degeneration.

The current standard treatment for macular degeneration is Laser Photocoagulation. An ophthalmologist performing laser photocoagulation locates the aberrant vessels with fluorescent angiography and selectively burns the vessels with the laser ablation technique. A side effect of laser surgery is the destruction of the retinal layer immediately overlying the aberrant vessels. Patients treated with laser photocoagulation have a measurable loss of vision immediately after treatment and this is an unacceptable negative side effect. Overall, laser surgery is viewed as a stopgap treatment that is only moderately effective at slowing the disease.

Photodynamic therapy is the current state of the art treatment for macular degeneration. The U.S. Food and Drug Administration approved verteporfin for injection (Visudyne™ developed by Ciba Vision & QLT) to treat the wet form of age-related macular degeneration. A patient being treated with photodynamic therapy is injected with the photo-reactive compound (verteporfin) and immediately treated with a non-destructive ophthalmic laser. The ophthalmologist performing the surgery identifies the aberrant vessels and directs the laser beam toward the aberrant vessels. Verteporfin, when activated by the laser, generates a transient burst of energy that effectively scorches any cells within the vicinity of the activated molecule. (Source: HHS News, U.S. Dept. of Health and Human Services, Apr. 13, 2000)

Ionizing radiation is used to kill proliferating vessels (proliferating cells are more sensitive to radiation than quiescent cells). Ionizing radiation is usually administered in a beam large enough to expose most of the eye. In 1993, a group at the University of Belfast in Northern Ireland reported that they had tried X-rays on a small number patients with the wet form of macular degeneration. Their positive results have been supported by several similar studies with X-rays done by other research teams in Europe.

Another debilitating ocular disease is Retinopathy of Prematurity (ROP). ROP is an eye disease that occurs in a significant percentage of premature babies. The last 12 weeks of a full-term delivery (weeks 28 to 40) are particularly active months in the development of the fetal eye. The pre-natal development of the retinal blood supply (choroid) initiates at the optic nerve on week 16 and progresses in a radial fashion towards the anterior region of the retina until birth (week 40). If birth is premature, the retinal vasculature does not have enough time to fully develop and the anterior edges of the retina become deprived of oxygen. The lack of anterior-retinal oxygenation is the underlying cause of ROP. (Source: The Association of Retinopathy of Prematurity and Related Diseases, Franklin, Mich.)

In premature infants a significant portion of the anterior retina is deprived of an adequate blood supply. The oxygen deprived anterior retina responds by signaling for the growth of new vessels. Abnormal neovascularization in the zone between the anterior and posterior retina initiates a cascade of events with severe pathologic consequences. As new vessels grow in response to the chemical signals, arterio-venous shunts are formed in the zone between vascularized posterior retina and the avascular anterior retina. These vascular shunts gradually enlarge, becoming thicker and more elevated. The new vessels are accompanied by infiltrating fibroblasts, which produce fibrous scar tissue. Eventually, a ring of scar tissue is formed which is attached to the retina as well as to the vitreous gel. The ring of scar tissue may extend for 360 degrees around the inside of the eye. When this scar tissue contracts it pulls the retina and produces a retinal detachment. If enough scar tissue forms, the retina can become completely detached. Premature neonates are at risk for developing ROP because they have been taken out of the protective environment of the uterus and are exposed to a variety of angiogenic stimuli, including medications, high levels of oxygen, and variations in light and temperature. Some or all of these factors may have an effect on the development of ROP. Fortunately, most premature infants do not develop ROP, and most infants with ROP improve spontaneously. If ROP does develop, it usually occurs between 34 and 40 weeks after conception, regardless of gestational age at birth.

A technique termed cryotherapy has been shown to have a beneficial effect for the treatment of ROP. Cryotherapy involves placing a sub-zero probe on the outer wall of the eye (sclera). The probe causes a zone of ice crystallization on the retinal surface between the sclera and the vitreous. Multiple applications of cryotherapy are performed in order to treat the entire avascular area, which is anterior to the neovascular ridge. Treatment of the ridge itself is avoided, since the ridge tends to bleed and cause vitreous hemorrhage if frozen.

The mechanism of action of cryotherapy is not completely understood. The working hypothesis is that the cryotherapy probably damages the avascular anterior retinal layer. This damage results in a thinning of the retina which allows for facilitated diffusion of oxygen to the remaining viable cells. In addition, a cryo-treated retina has fewer viable cells and thus a reduced demand for oxygen. The reduced demand for oxygen dampens the angiogenic stimuli and halts the neovascularization. Cryotherapy was found to reduce the risk of retinal detachment from 43% in the untreated eyes to 21% in the treated eyes. Cryotherapy does, however, have potential complications; the procedure is often performed under general anesthesia which can be risky for premature infants.

Laser photocoagulation, described hereinabove, uses similar principles in the treatment of ROP. The laser treatment is applied to the anterior retina that does not yet have a blood supply. The purpose of the treatment is to eliminate the abnormal vessels before they lay down enough scar tissue to produce a retinal detachment. In addition, the avascular anterior retina is marginally thinned by the laser reducing the need for oxygen and dampening the angiogenic stimuli, much like cryotherapy. Laser therapy is superior to cryotherapy in that it is directed at the retina and not the entire thickness of the eye wall. Because laser therapy involves less tissue and is not painful, post-treatment inflammation is greatly reduced. When compared to cryotherapy, laser therapy is superior because there is a reduced need for anesthetics.

If laser therapy or cryotherapy is unsuccessful in halting the progression of ROP, some surgical treatments are available. If there is a shallow retinal detachment due to a small traction from the fibro-vascular scar tissue, a procedure called scleral buckling may be of benefit. Scleral buckling involves placing a silicone band around the equator of the eye and tightening it to produce a slight indentation on the inside of the eye. This band relieves the traction of the vitreous gel pulling on the fibrous scar tissue and the retina. This allows the retina to flatten onto the wall of the eye and resume normal function. Infants who have had scleral buckling may maintain good vision in the eye, particularly if the macula did not detach. The encircling band usually needs to be removed some months or years later because the eye will continue to grow, producing gradually increasing compression of the globe and induced nearsightedness.

In late stage ROP, with complete retinal detachment due to scar tissue on the retina, scleral buckling is not sufficient to relieve the traction. For these infants, a vitrectomy may be considered. Vitrectomy involves making several small incisions into the eye, and using a suction/cutter device to remove the vitreous gel. The vitreous is replaced with a saline solution to keep the eye formed, and the eye is able to maintain its shape and pressure indefinitely without the vitreous gel. After the vitreous has been removed, the scar tissue on the retina can be peeled or cut away, allowing the retina to relax and lay back down against the eye wall. It may take some weeks for the retina to become re-attached after the surgery, and if holes or tears in the retina occur during the procedure, the retina usually will not re-attach. The lens of the eye often has to be removed to allow complete dissection of the scar tissue, but some newer techniques are being tried that can preserve the lens.

The success rate for vitrectomy surgery for ROP is, however, somewhat limited. The published anatomic success rate, which means getting the retina reattached to the wall of the eye, ranges from 25% to 50% of patients undergoing surgery. The functional success rate, which means the ability to see well, is significantly lower. Of eyes that have “successful” vitrectomy surgery (anatomic success), only about ¼ are able to see well enough to reach out and grab an object or recognize patterns.

Another debilitating ocular disease occurs in patients who suffer from diabetes mellitus. Approximately 14 million Americans have diabetes mellitus. In addition to causing numerous systemic complications (such as kidney failure, hypertension, and cardiovascular disease), diabetes is one of the leading causes of blindness among working-age Americans. In fact, the risk of blindness to persons with diabetes is 25 times greater than that of the general population. Many patients with diabetic eye problems are asymptomatic despite the presence of vision-threatening disease. If diabetic eye disease is left untreated, it can lead to serious visual loss. Decreased vision due to diabetes can be caused by several mechanisms, and treatment needs to be tailored to the individual's needs. (Source: The Center for Disease Control, “The Prevention and Treatment of Diabetes Mellitus—A Guide for Primary Care Practitioners”: www.cdc.gov/health/diseases.htm)

Many diabetics notice blurred vision when their blood sugar is particularly high or low. This blurred vision results from changes in the shape of the lens of the eyes, and usually reverse when their blood sugar returns to normal. Diabetes is a disease that affects not only the patient's blood sugar levels, but also the blood vessels. Symptoms associated with diabetes (including elevated blood pressure) cause damage to the microcirculatory system including the capillaries associated with the retina. Capillary damage results in a decreased flow of blood to isolated regions of the retina. In addition, the damaged blood vessels tend to leak, which produces swelling within the retina.

There are two main categories of diabetic eye disease. The first category is termed background diabetic retinopathy or non-proliferative retinopathy. This is essentially the earliest stage of diabetic retinopathy. This stage is characterized by damage to small retinal blood vessels which results in the effusion of fluid (blood) into the retina. Most visual loss during this stage is due to the fluid accumulating in the macula. This accumulation of fluid is called macular edema, and can cause temporary or permanent decreased vision. The second category of diabetic retinopathy is termed proliferative diabetic retinopathy. Proliferative retinopathy is the end result of diabetes-induced damage sustained by the retinal capillary bed (choroid). Damage to the choroid causes oxygen deprivation in the retina. The retinal tissue responds to its anoxic environment by producing angiogenic cytokines that stimulate neovascularization. As was previously stated, neovascularization of the retina causes bleeding in the eye, retinal scar tissue, retinal detachments, and any of one of these symptoms can cause decreased vision or blindness. Diabetics often also suffer from neovascular glaucoma, which manifests in rubeosis, blood vessels growing on the iris that causes closure of the angle.

Diabetic retinopathy can occur in both Type I diabetics (onset of diabetes prior to age 40) and Type II diabetics (onset after age 40), although it tends to be more common and more severe in Type I patients. Because Type II diabetes is often not diagnosed until the patient has had the disease for many years, diabetic retinopathy may be present in a Type II patient at the time diabetes is discovered.

The treatment of diabetic retinopathy depends upon multiple factors, including the type and degree of retinopathy, associated ocular factors such as cataract or vitreous hemorrhage, and the medical history of the patient. Treatment options include the same options that were discussed for ROP, namely laser photocoagulation, cryotherapy (freezing), and vitrectomy surgery. Blindness due to diabetic retinopathy is preventable in most cases.

Intraocular cancerous tumors of any type are mostly uncommon. Ocular tumors are, however, extremely serious in that uveal (eye) cancers generally metastasize to and from other areas of the body. The most common primary malignant tumor of the eye, uveal melanoma, occurs in 7 persons per million in the general population per year—less than one tenth the incidence of lung cancer. Retinoblastoma occurs as a childhood disease approximately as frequently as hemophilia. These two intraocular tumors are very different and related only by anatomic proximity. The choice of treatment for ocular cancer depends on where the cancer is in the eye, how far it has spread, and the patient's general health and age. (Source: The Eye Cancer Network: eyecancer.com; OncoLink: cancer.med.upenn.edu)

Retinoblastoma is a cancer of one or both eyes which occurs in young children. There are approximately 350 new diagnosed cases per year in the Unites States. Retinoblastoma affects one in every 15,000 to 30,000 live babies that are born in the United States. Retinoblastoma affects children of all races and both boys and girls.

The retinoblastoma tumor(s) originate in the retina, the light sensitive layer of the eye which enables the eye to see. The treatment of retinoblastoma is individualized for each patient and depends upon the age of the child, the involvement of one or both eyes, and whether or not the cancer has spread to other parts of the body. If left untreated, the child could die. Treatments for retinoblastoma include enucleation, external beam radiation, radioactive plaques, laser therapy, cryotherapy and chemoreduction.

Enucleation is the most common form of treatment for retinoblastoma. During an enucleation, the eye is surgically removed. This is necessary because it is the only way to remove the cancer completely. It is not possible to remove the cancer from within the eye without removing the entire eye. Although partial enucleation is possible for some other eye cancers, it is risky and may even contribute to the spread of the cancer for retinoblastoma patients.

When both eyes are involved, sometimes the more involved or “worse” eye is enucleated, while the other eye may be treated with one of the vision-preserving treatments, such as external-beam radiation, plaque therapy, cryotherapy, laser treatment, and chemoreduction which are described below.

External beam radiation has been used since the early 1900's as a way to save the eye(s) and vision. Retinoblastoma is sensitive to radiation, and frequently the treatment is successful. The radiation treatment is performed on an outpatient basis five times per week over a 3 to 4 week stretch. Custom-made plaster-of paris molds are made to prevent the head from moving during treatment and sometimes sedatives are prescribed prior to treatment.

Tumors usually get smaller (regress) and look scarred after external beam radiation treatment but they rarely disappear completely. In fact, they may even become more obvious as they shrink, because the pinkish-grey tumor mass is replaced by white calcium. Immediately after treatment, the skin may be sunburned or a small patch of hair may be lost in the back of the head from the beam exit position. Following external beam radiation, long-term effects can include cataracts, radiation retinopathy (bleeding and exudates of the retina), impaired vision, and temporal bone suppression (bones on the side of the head which do not grow normally). Radiation can also increase a child's risk of developing other tumors outside the eye for those children who carry the abnormal gene in every cell of their bodies.

Radioactive plaques are disks of radioactive material that were developed in the 1930's to radiate retinoblastoma. Today, the isotope iodine-125 is used and the plaques are custom-built for each child. The child must generally be hospitalized for this procedure, and undergoes two separate operations (one to insert the plaque and one to remove it) over 3 to 7 days.

Laser therapy, sometimes referred to as photocoagulation or laser hyperthermia (which are two different techniques), is a non-invasive treatment for retinoblastoma. Lasers effectively destroy smaller retinoblastoma tumors. This type of treatment is usually performed by focusing light through the pupil onto and surrounding the cancers in the eye. Recently a new delivery system of the laser, called a diopexy probe, has enabled treatment of the cancer by aiming the light through the wall of the eye and not through the pupil. Laser treatment is done under local or general anesthesia, usually does not have any post-operative pain associated with it, and does not require any post-operative medications. Laser can be used alone or in addition to external-beam radiation, plaques, or cryotherapy.

Cryotherapy may also be performed on patients suffering from retinoblastoma. Cryotherapy is performed under local or general anesthesia and freezes smaller retinoblastoma tumors. A pen-like probe is placed on the sclera adjacent to the tumor and the tumor is frozen. Cryotherapy usually has to be repeated many times to successfully destroy all of the cancer cells. An adverse side effect of cryotherapy is that it causes the lids and eye to swell for 1 to 5 days; sometimes the swelling is so much that the children are unable to open their lids for a few days. Eye drops or ointment is often given to reduce the swelling.

Chemoreduction is the treatment of retinoblastoma with chemotherapy. Chemotherapy is generally administered intravenously to the child, passes through the blood stream, and causes the tumors to shrink within a few weeks if successful. Chemotherapy, with one or more drugs, can be given once, twice, or more. Depending on the drug(s) and on the institution, the child may or may not be hospitalized during this process. After chemotherapy, the child is re-examined and the remaining tumor(s) are treated with cryotherapy, laser, or radioactive plaque. Children may require as many as twenty treatments with re-examinations of the eye under anesthesia every 3 weeks.

Although it is rare if the retinoblastoma is treated promptly, retinoblastoma can spread (metastasize) outside of the eye to the brain, the central nervous system (brain and spinal cord), and the bones. In this case, chemotherapy is prescribed by a pediatric oncologist and is administered through the peripheral blood vessels or into the brain for months to years after initial diagnosis of metastatic disease.

Tumors other than retinoblastoma and melanoma occur in the eye, and they are often the harbingers of disease elsewhere. Choroidal metastasis is the most frequently occurring intraocular malignancy and can be the initial manifestation of systemic malignancy. Choroidal metastases resemble nonpigmented melanomas. They have a similar appearance to melanoma on fluorescein angio-gram and show subtle echographic differences on ultrasonograms. Choroidal metastases, however, grow more rapidly and are more likely to cause large exudative retinal detachments.

In general, the prognosis for survival is poor once metastatic disease is found in the eye. As survival in systemic cancer patients improves, however, successful treatment of ocular metastases has an increasingly important role in maintaining a good quality of life.

Primary ocular lymphoma is one of the most intriguing intraocular tumors. Its relationship with primary central nervous system lymphoma and the propensity of the tumor to proliferate in the subretinal pigment epithelial space, where no lymphoid tissue exists, are just two fascinating aspects of this highly aggressive lymphoma. The clinical manifestations of primary ocular lymphoma are notorious for mimicking benign uveitic entities and thus delaying the correct diagnosis for months. The neoplastic cells in ocular lymphoma can remain confined to the space between the retinal pigment epithelium and Bruch's membrane. Because the vitritis associated with these aggregates of lymphoma often consists of reactive lymphocytes, vitreous biopsy can be nondiagnostic. This has lead to the misconception that it is difficult to interpret intraocular cytology, when, in fact, surgeons were not harvesting tumor cells. The positive yield from intraocular biopsy can be increased in some cases if the surgeon performs an aspiration biopsy via retinotomy in the subretinal pigment epithelial space. Primary ocular lymphoma consists of large, cytologically atypical cells that stain positive for leukocyte common antigen. Aspirates are usually associated with large amounts of necrotic debris. Immunophenotypic analysis has been problematic in the past. Some early studies failed to find any surface markers and concluded that ocular lymphoma was a null-cell tumor. Pretreatment of cells with hyaluronidase has increased the yield of immuno-pathologic studies.

Another form of ocular cancer is choroidal melanoma. Choroidal melanoma is a primary cancer of the eye. It arises from the pigmented cells of the choroid of the eye and is not a tumor that started somewhere else and spread to the eye. Although some choroidal melanomas are more life-threatening than others, almost all should be treated as if they were malignant. Some choroidal melanomas appear to remain dormant and do not grow. Most enlarge slowly over time and lead to loss of vision. These tumors can spread to other parts of the body and lead eventually to death. Numerous cases have been reported of ocular melanoma metastasizing to the liver. (Source: The Eye Cancer Network: www.eyecancer.com)

For many years, the usual treatment for choroidal melanoma has been enucleation. If the tumor has not spread to other parts of the body, removal of the eye generally rids the patient of the tumor completely. Since World War II, radiation treatment has been used for choroidal melanoma. During the past 20 years, this method of treatment has been refined. Radiation, at the appropriate dose rates and in the proper physical forms, is intended to eliminate growing tumor cells without causing damage to normal tissue sufficient to require removal of the eye. As the cells die, the tumor shrinks, but it usually does not disappear entirely. The most promising widely available method for irradiating medium choroidal melanoma involves constructing a small plaque with radioactive pellets glued to one side. Radiation, however, is usually accompanied by adverse side effects such as emesis and alopecia.

High-energy particles (helium ion or proton beam radiation) from a cyclotron can also be used to irradiate tumors. Surgery is performed first to sew small metal clips to the sclera so that the particle beam can be aimed accurately. Treatment is given over several successive days. The equipment needed for these treatments is available only in a few medical centers in the world. Good results have been reported in some patients, but many patients treated in this way have been followed for only a few years. Therefore, the long-term results of these forms of radiation therapy compared with the more commonly used plaque are unknown.

Over the years, other treatments have been used for a small number of patients. Photocoagulation using white light or laser light has been used to burn small tumors, and cryo-therapy has been used to kill the tumors by freezing them. These techniques are believed to work only for very small tumors. Some doctors have combined laser or cryotherapy with radiation, but such treatments are experimental. A few patients have had eye wall resection or a related procedure to remove tumors from their eyes. These methods of treatment are considered experimental by most doctors and have been used only for a small number of tumors. No treatment is available that can guarantee to destroy the tumor, to preserve vision, or to assure a normal lifespan.

Another ocular cancer is intraocular melanoma, a rare cancer in which cancer cells are found in the part of the eye called the uvea. The uvea contains cells called melanocytes, which contain pigment. When these cells become cancerous, the cancer is referred to as a melanoma. The uvea includes the iris (the colored part of the eye), the ciliary body (a muscle in the eye), and the choroid (a layer of tissue in the back of the eye). The iris opens and closes to change the amount of light entering the eye. The ciliary body changes the shape of the lens inside the eye so it can focus. The choroid layer is next to the retina, the part of the eye that makes a picture. If there is melanoma that starts in the iris, it may look like a dark spot on the iris. If melanoma is in the ciliary body or the choroid, a person may have blurry vision or may have no symptoms, and the cancer may grow before it is noticed. (Source: The Eye Cancer Network: www.eyecancer.com)

The chance of recovery (prognosis) from intraocular melanoma depends on the size and cell type of the cancer, where the cancer is in the eye, and whether the cancer has spread. There are treatments for all patients with intraocular melanoma. Three types of treatment are commonly administered, namely surgery (removal of the cancer), radiation therapy (using high-dose x-rays or other high-energy rays to “kill” the cancer cells), and photocoagulation (destroying blood vessels that feed the tumor).

Surgery is the most common treatment of intraocular melanoma. A doctor may remove the cancer using one of the following operations:

Iridectomy—removal of only parts of the iris;

Iridotrabeculectomy—removal of parts of the iris and the supporting tissues around the cornea, the clear layer covering the front of the eye;

Iridocyclectomy—removal of parts of the iris and the ciliary body;

Choroidectomy—removal of parts of the choroids;

Enucleation—removal of the entire eye.

Radiation therapy can also be used to apply x-rays or other high-energy rays to the area where the cancer cells exist so as kill cancer cells and shrink the tumors. Radiation can be used alone or in combination with surgery. Photocoagulation treatment may also be used wherein a tiny beam of light, usually from a laser, is applied to the eye to destroy blood vessels and kill the tumor.

The overwhelming majority of proposed therapies for the treatment of ocular disease, particularly subretinal neovascularization and ocular tumors, initially employ surgery or radiation treatment. When patients are treated with medication, alone or following, surgery, the administration of the medication is generally systemic, either via injection or orally. As noted previously, surgery and radiation treatment for ocular diseases are both painful, often require long recovery periods, and may be followed by adverse side effects. Additionally, systemic administration via oral ingestion of a drug or injection at a site other than the eye are often provided in ineffective amounts, necessitating administration of often unacceptably high levels of the drug in order to achieve effective intraocular concentrations. There is thus a major need for a successful non-systemic therapy for the treatment of ocular diseases, such as corneal and retinal neovascularization. Additionally, delivery of drugs and medicaments to the eye without adverse side effect remains a major challenge. The subject invention provides such a therapy, providing for efficacious non-systemic administration of a tubulin binding agent for the treatment of ocular disease, with minimal side effects.

### SUMMARY OF THE INVENTION

The present invention is directed to the administration of a vascular targeting agent (“VTA”), particularly a tubulin binding agent, for the treatment of malignant or non-malignant vascular proliferative disorders in ocular tissue.

Neovascularization of ocular tissue is a pathogenic condition characterized by vascular proliferation and occurs in a variety of ocular diseases with varying degrees of vision failure. The administration of a VTA for the pharmacological control of the neovascularization associated with non-malignant vascular proliferative disorders such as wet macular degeneration, proliferative diabetic retinopathy or retinopathy of prematurity would potentially benefit patients for which few therapeutic options are available. In another embodiment, the invention provides the administration of a VTA for the pharmacological control of neovascularization associated with malignant vascular proliferative disorders such as ocular tumors.

The blood-retinal barrier (BRB) is composed of specialized nonfenestrated tightly-joined endothelial cells that form a transport barrier for certain substances between the retinal capillaries and the retinal tissue. The nascent vessels of the cornea and retina associated with the retinopathies are aberrant, much like the vessels associated with solid tumors. Tubulin binding agents, inhibitors of tubulin polymerization and vascular targeting agents, may be able to attack the aberrant vessels because these vessels do not share architectural similarities with the blood retinal barrier. Tubulin binding agents may halt the progression of the disease much like they do with a tumor-vasculature. Local (non-systemic) delivery of tubulin binding agents to the eye can be achieved using intravitreal injection, sub-Tenon's injection, ophthalmic drops iontophoresis, and implants and/or inserts. Systemic administration may be accomplished by administration of the tubulin binding agents into the bloodstream at a site which is separated by a measurable distance from the diseased or affected organ or tissue, in this case they eye. Preferred modes of systemic administration include parenteral or oral administration.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference.

### DETAILED DESCRIPTION OF THE DRAWINGS

The invention will be better understood by reference to the appended figures of which:

FIG. 1 is a simplified front and side anatomic illustration of a mammalian eye;

FIG. 2A illustrates normal macula;

FIG. 2B illustrates dry-form macular degeneration;

FIG. 2C illustrates wet-form macular degeneration;

FIGS. 3A and 3B are magnified photographs of a portion of the cornea showing the inhibition of vessel growth on Day 28 following in administration of CA4P administration in comparison with a vehicle control eye; and

FIGS. 4A and 4B illustrate microscopic histology of changes to the cornea (inhibition of vessel growth) on Day 28 following systemic administration of CA4P in comparison with a vehicle control eye.

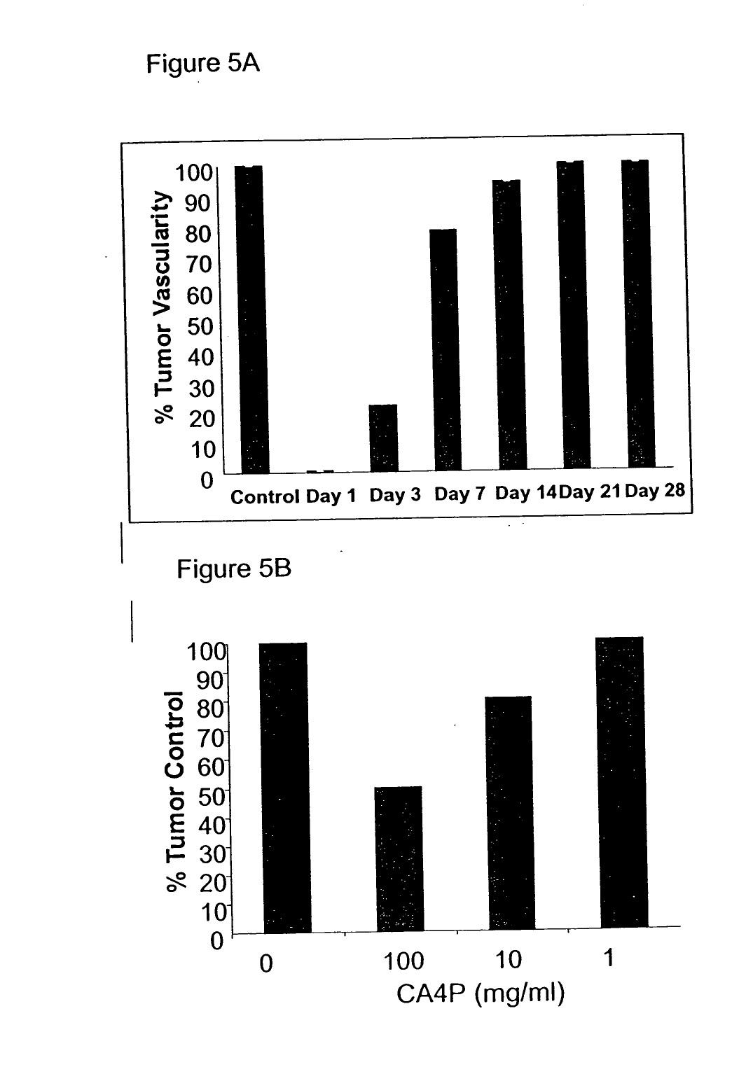

FIG. 5A illustrates the effect of a single dose of CA4P the vascularization of an ocular tumor in an animal model of retinoblastoma.

FIG. 5B illustrates the degree of tumor regression in an animal model of retinoblastoma following repetitive dosing of CA4P.

### DETAILED DESCRIPTION OF THE INVENTION

The present invention is directed to methods and compositions for the treatment or prevention of ocular disease in a subject. The method comprises the steps of preparing a dosage comprising a pharmaceutically effective dosage of a tubulin binding agent and administering the pharmaceutically effective dosage to a subject in need thereof.

One embodiment is a method of treating or preventing ocular diseases by administering a tubulin binding agent to the eye of a subject in need thereof in a dose sufficient to achieve a concentration of the tubulin binding agent in the eye in the range between approximately 1 nM to approximately 100 mM of aqueous humour tissue.

Another method of the present invention method is the administration of a tubulin binding agent to a subject in need thereof in a dose sufficient to reduce the leakage of exudate from a lesion in the eye of a subject having choroidal neovascularization and identified as having a lesion.

Another method of the present invention is the administration of a tubulin binding agent to a subject in need thereof in a dose sufficient to induce regression of proliferating vasculature in the eye of a subject suffering from choroidal neovascularization.

The present invention is also directed to a pharmaceutical medicament for the treatment or prevention of ocular disease, comprising a therapeutically effective amount of a tubulin binding agent for reducing ocular neovascularization in association with a pharmaceutically acceptable carrier, excipient, diluent or adjuvant for administration to a subject in need thereof.

The subject is preferably a mammal, more preferably a human. Preferred tubulin binding agents for the compositions and methods of the present invention include combretastatin A4 and combretastatin A4 prodrug.

Ocular diseases treated or prevented by the present compositions and methods include neovascularization of the retina, neovascularization of the choroid, neovascularization of ocular tumors, diabetic retinopathy, retinopathy of prematurity, retinoblastoma, neovascularization of the cornea, and macular degeneration. More specifically, suitable diseases include those which exhibit subfoveal choroidal neovascularization, including pathological myopia and exudative age-related macular degeneration. Pathological myopia can be referred to alternately as proliferative myopathy or myopic macular degeneration. As used herein, the terms pathological myopia, proliferative myopathy and myopic macular degeneration all refer to the same disease state. Ocular tumors may include retinoblastoma, primary ocular lymphoma, choroidal melanoma, and intraocular melanoma.

The tubulin binding agent may be delivered either systemically or non-systemically. Preferred embodiments of non-systemic administration include intravitreal injection, sub-conjunctival injection, peri-ocular injection, sub-Tenon's injection, ophthalmic drops, iontophoresis and ocular implant and/or ocular insert. A suitable dosage range for tubulin binding agents administered non-systemically is in the range of from approximately 0.1 mg/ml to approximately 100 mg/ml.

Preferred embodiments of systemic administration include parenteral and oral. More specific systemic routes of administration include intravenous, intradermal, intramuscular, subcutaneous, inhalation, transmucosal, and rectal. A suitable dosage range for tubulin binding agents administered systemically is in the range of from approximately 0.1 mg/m² to approximately 120 mg/m². Preferred dosage ranges include from approximately 2 mg/m² to approximately 90 mg/m², approximately 15 mg/m² to approximately 50 mg/m², approximately 10 mg/m² to approximately 80 mg/m², and approximately 20 mg/m² to approximately 60 mg/m². A particularly preferred dosage for tubulin binding agents administered systemically, the dosage range used to treat the patient described in Example 9, is 27 mg/m². When the tubulin binding agent is a phosphate prodrug, the dosage is calculated based on the amount of free acid of the phosphate.

A preferred embodiment of a pharmaceutical composition of the present invention comprises in a suspension, emulsion or solution an amount of CA4P in the range of from approximately 0.1 mg/ml to approximately 100 mg/ml; approximately 5 mg/ml carboxymethylcellulose; and approximately 9 mg/ml NaCl. This composition preferably has a final pH in the range of from approximately 6.6 to 8.6, osmolarity in the range of from approximately 291-492 mosmol/kg H₂O and viscosity in the range of from approximately 50-66 mPa.s. The human eye possess several structurally unique properties: it is exposed to the environment, it is highly enervated, it has a high rate of blood flow in the choroid yet the anterior chamber and vitreous humor are completely avascular and isolated from the circulatory system. The exceptional architecture of the eye provides ample opportunity for delivery of tubulin binding agents by one or more non-systemic methods of administration for the treatment of ocular conditions, diseases, tumors and disorders. A simplified anatomic illustration of the eye is shown in FIG. 1.

As recited previously, neovascularization of ocular tissue is a pathogenic condition that occurs in a variety of ocular diseases and is associated with varying degrees of vision failure. Pharmacological control of neovascularization would potentially benefit patients suffering from diseases such as wet macular degeneration, proliferative diabetic retinopathy and retinopathy of prematurity.

Tubulin binding agents inhibit tubulin assembly by binding to tubulin-binding cofactors or cofactor-tubulin complexes in a cell during mitosis and prevent the division and thus proliferation of the cell. Tubulin binding agents comprise a broad class of compounds which inhibit tubulin polymerization, and which generally function as tumor selective vascular targeting agents useful for cancer chemotherapy, as well as for other non-cancer applications such as ocular disease.

As discussed above, one of the disadvantages of systemic administration of drugs for treating ocular diseases is that systemic administration does not generally provide effective levels of the drug specifically to the eye. Since drugs administered systemically may be metabolized in the body before even reaching the eye, higher levels of the drug may need to be administered in order to achieve effective intraocular concentrations. Non-systemic or local administration of drugs directly to the eye(s) of a patient suffering from an ocular disease allows the effective concentration of drug to be administered and benefits the patient immeasurably.

Ocular indications treatable by the non-systemic or systemic administration of the tubulin binding agents in accordance with the present invention include non-malignant vascular proliferative diseases characterized by corneal, iris, trabecular meshwork, retinal, subretinal, optical nerve head, or choroidal neovascularization, as well as malignant vascular proliferative diseases such as ocular tumors and cancers.

Corneal neovascularization occurs in the following: trachoma (Chlamydia trachomatis), viral interstitial keratitis, microbial keratoconjunctivitis, corneal transplantation and burns. It may be caused by infection (trachoma, herpes, leishmaniasis, onchoceroiasis), transplantation, burns (heat, alkalai), trauma, nutritional deficiency and contact lens induced damage. Diseases involving iris neovascularization include rubeosis iritis, Fuchs' heteochromic iridocyclitis, and developmental hypoplasia of the iris.

Retinal and/or choroidal neovascularization occurs in macular degeneration, diabetic retinopathy, sickle cell retinopathy, and retinopathy of prematurity. Choroidal neovascularization occurs when vessels from the choroidal membrane grow through a break in Bruch's membrane and into the subretinal pigment epithelium or the subretinal space, manifesting as fluid accumulation (edema) and or hemorrhaging. This in itself can lead to severe vision loss, however the retinal pigment epithelium or the neurosensory retina may also detach. In a preferred embodiment, the invention involves the treatment of highly proliferative subfoveal choroidal neovascularization which occurs as a result of or concurrent with exudative (wet) forms of age-related macular degeneration, diabetic retinopathy, retinopathy of prematurity, pathologic myopia, posterior uveitis, chronic uveitis, ocular histoplasmosis syndrome, macular edema, retinal vein occlusion, angiod streaks, choroidal rupture, multifocal choroiditis, ischemic retinal disease, and other uveitic entities.

A particularly preferred form of subfoveal choroidal neovascularization occurs as a result of or concurrent with pathological myopia. High myopia (extreme near sightedness) is a condition in characterized by abnormal growth of the eyeball causing stretching of the retina and Bruch's membrane. A gradual decrease in vision occurs when the macula is thinned as a result of the retinal stretching. The thinning of Bruch's membrane can result in cracks through which neovasculature can grow from the choroid underneath the retina. Subfovial choroidal neovascularization can cause sudden and severe loss of vision. Another particularly preferred form of subfoveal choroidal neovascularization occurs as a result of, or concurrent with, exudative age-related macular degeneration. Anterior chamber neovascularization occurs in neovascular glaucoma.

Among the non-systemic methods of administering tubulin binding agents contemplated by the present invention are: intravitreal administration (injection), sub-conjunctival administration, peri-ocular administration, sub-Tenon's injection, iontophoretic delivery, topical administration with ophthalmic drops, gels, or ointments, and via ocular insert or implant.

Tubulin binding agents may be administered intravitreally via an injection directly into the vitreous humor of the eye. Tubulin binding agents may also be administered beneath the conjunctiva by sub-conjunctival injection, and around the eye via peri-ocular injection.

Tubulin binding agents may also be administered by injection into the sub-Tenon's space (under Tenon's capsule) with a blunt tip Connor Cannula. Using proper technique, the medical professional administering the dosage of tubulin binding agent can avoid puncturing the globe and damaging the optic nerve. After delivery, the injection site is cauterized and the space serves as a depot for the drug. Administration into the sub-Tenon's space is less invasive than intravitreal injection.

In another embodiment of the present invention, a tubulin binding agent may be formulated as a biocompatible, biodegradable, and/or bioerodible ocular implant or insert containing the tubulin binding agent so as to provide slow release of the drug and maintenance of a therapeutically effective drug concentration for an extended period of time. Drug-containing bioerodible ocular implants for implantation or insertion into a mammalian eye are described, for example, in U.S. Pat. No. 5,904,144 and U.S. Pat. No. 5,766,242, which are incorporated by reference herein in its entirety. Ocular implants generally comprise a capsule that is placed in a desired location in the eye. The capsule may include one or more medicaments or may include cells that produce a biologically active molecule for continuous, controlled delivery to the eye. The amount of drug that may be employed in this embodiment will vary depending on the effective dosage of the drug and the rate of release from the insert or implant on or within the eye.

Because the sclera is exposed, an iontophoretic probe may be applied onto the surface of the eye. lontophoresis uses an electrical current to drive the flux of ionic compounds across a cell membrane. This technique is currently utilized for transdermal delivery of ionic drugs. The two principal mechanisms by which iontophoresis drives the transport of drugs are: (a) iontophoresis, in which a charged ion is repelled from an electrode of the same charge, and (b) electroosmosis, the convective movement of solvent that occurs through a charged “pore” in response to the preferential passage of counter-ions when the electric field is applied.

The tubulin binding agents may also be formulated for topical administration to the eye in the form of sterile, ophthalmic drops.

In accordance with the present invention, the preferred tubulin binding agent is combretastatin A4 (“CA4”), a potent vascular targeting agent. CA4 is essentially insoluble in water. This characteristic interferes with the formulation of pharmaceutical preparations of this compound. Thus, the more preferable prodrug form of combretastatin A4 (“CA4P”) is utilized to compensate for the generally poor solubility of CA4. As used herein, CA4P refers to all prodrug salts of combretastatin A4. Suitable CA4P salts include, inter alia, the phosphate prodrug described in U.S. Pat. No. 5,561,122 and the TRIS prodrug described in WO 02/22626. The invention is not limited in this respect, however, and formulations of CA4 may work as well or better than CA4P.

Combretastatins are derived from tropical and subtropical shrubs and trees of the Combretaceae family, which represent a practically unexplored reservoir of new substances with potentially useful biological properties. Illustrative is the genus Combretum with 25 species (10% of the total) known in the primitive medical practices of Africa and India for uses as diverse as treating leprosy (See: Watt, J. M. et al, “The Medicinal and Poisonous Plants of Southern and Eastern Africa”, E. & S. Livingstone, Ltd., London, 1962, p. 194) (Combretum sp. root) and cancer (Combretum latifolium).

Combretastatins have been found to be antineoplastic substances. Numerous combretastatins have been isolated, structurally elucidated and synthesized. U.S. Pat. Nos. 5,409,953 and 5,59,786 describe the isolation and synthesis of Combretastatins designated as A-1, A-2, A-3, B-1, B-2, B-3 and B-4. The disclosures of these patents are incorporated by reference herein in their entirety. A related Combretastatin, designated Combretastatin A4, was described in U.S. Pat. No. 4,996,237 to Pettit, and which is incorporated by reference herein in its entirety.

CA4P is a derivative of the natural combretastatin A4 subtype described in U.S. Pat. No. 5,561,122, the entire disclosure of which is incorporated by reference herein. The preferred CA4P compound substitutes a disodium phosphate derivative for the —OH group in the CA4 structure and which allows metabolic conversion of CA4P back into the water insoluble CA4 in vivo. The invention is not, however, limited to the phosphate derivative, and other prodrug moieties may be substituted for the —OH group in the CA4 compound. In addition, phosphate prodrug salts other than the disodium salt of CA4P are expected to perform in substantially the same way for the purposes of this invention. Examples of other phosphate prodrug salts, including TRIS salts, are described in PCT patent applications WO 02/22626 and WO 99/35150, the disclosures of which are incorporated herein.

CA4P is the first in a new class of drugs—anti-tumor vascular targeting agents—that shrink solid tumors by selectively targeting and destroying the tumor-specific blood vessels formed by angiogenesis. Anti-tumor vascular targeting and angiogenesis inhibition are related cancer therapies that radically depart from conventional approaches to treating cancer. In contrast to traditional methods involving a direct attack on cancer cells, these new drugs target a tumor's life support system, the network of newly emerging blood vessels that form as a result of angiogenesis, the sprouting of new blood vessels from previously existing ones. Preclinical studies have shown that the use of these therapies can cause a tumor to shrink and ultimately disappear. Additionally, when CA4P was used in in vitro and in vivo animal cell models, it displayed a remarkable specificity for vascular toxicity (Int. J. Radiat. Oncol. Biol. Phys. 42 (4): 895-903, 1998; Cancer Res. 57(10): 1839-1834 1997).

While angiogenesis inhibitors and anti-tumor vascular targeting agents, such as combretastatin, both target a tumor's blood vessels, they differ in their approach and in the end result. With angiogenesis inhibition, the aim is to prevent tumor growth by inhibiting the formation of tumor-specific blood vessels that feed and sustain the tumor. On the other hand, with anti-tumor vascular targeting the goal is to obliterate tumors by selectively attacking and destroying their existing blood vessels, creating a rapid and irreversible shutdown of these blood vessels. Such an effect is not observed with anti-angiogenesis drugs. Only antivascular targeting activity can destroy existing blood vessels supporting tumor growth. Combretastatin also has the ability to inhibit the proliferation of endothelial cells which produce and line new tumor vasculature (anti-angiogenic activity). Hence, it is thought that Combretastatin can behave both as a anti-tumor vascular targeting agent and as an anti-angiogenic drug. In preclinical studies, both therapies have been shown to leave blood vessels associated with normal tissue unaffected. The present invention contemplates the administration of CA4P both alone, and/or in combination with current state of the art medicaments for the treatment of ocular diseases.

Vasculature formed by angiogenesis has also been observed in diseases other than cancer including diseases of the eye, e.g. macular degeneration, proliferative diabetic retinopathy and retinopathy of prematurity. Preliminary work toward reducing such vasculature in an experimental eye model was carried out from the laboratory of Donald Armstrong, Ph.D., D.Sc., University of Florida, College of Veterinary Medicine, Division of Ophthalmology, who demonstrated that CA4P accelerated the regression rate of preformed vessels in the eye of experimental animal models. FIGS. 3A, 3B, 4A and 4B illustrate the regression of preformed vessels in the eyes of rabbits studied in this experiment.

CA4 and CA4P are currently undergoing clinical testing for treatment of a variety of diseases and indications including use as an anti-tumor vascular targeting agent, and as inhibitor of angiogenesis. Furthermore, CA4P has demonstrated the ability to treat ocular diseases, such as subretinal neovascularization.

The present invention also contemplates the use of synthetic analogs of the Combretastatins as described in Bioorg. Med. Chem. Lett. 11(2001) 871-874, 3073-3076, J. Med. Chem. (2002), 45: 1697-1711, WO 02/50007, WO 01/12579, WO 00/35865, WO 00/48590, WO 01/12579, U.S. Pat. No. 5,525,632, U.S. Pat. No. 5,674,906, and U.S. Pat. No. 5,731,353.

Other tubulin binding agents which may be administered as VTAs include the following agents or their prodrugs: 2,3-disubstituted Benzo[b]thiophenes (U.S. Pat. Nos. 5,886, 025; 6,162,930, and 6,350,777), 2,3-disubstituted benzo[b]furans (WO 98/39323), 2-3-disubstituted indoles (WO01/19794), disubstituted dihydronaphthalenes (WO01/68654), or Colchicine analogs (WO 99/02166). Furthermore, additional non-cytotoxic prodrugs of vascular targeting agents, which are converted to a substantially cytotoxic drug by action of an endothelial enzyme selectively induced at enhanced levels at sites of vascular proliferation are disclosed in WO00/48606.

Additional known tubulin binding agents which may be administered in accordance with the present invention include: taxanes, vinblastine (vinca alkaloids), colchicines (colchicinoids), dolastatins, podophyllotoxins, steganacins, amphtethiniles, flavanoids, rhizoxins, curacins A, ephothilones A and B, welwistatins, phenstatins, 2-strylquinazolin-4(3H)-ones, stilbenes, 2-aryl-1,8-naphthyridin-4(1H)-ones, and 5,6-dihydroindolo(2,1-a)isoquinolines.

With regard to the administration and delivery of the tubulin binding agents to the eye of a subject in need thereof, it is important to consider that the human eye possesses several structurally unique properties: it is exposed to the environment, it is highly enervated, it has a high rate of blood flow in the choroid yet the anterior chamber and vitreous humor are completely avascular and isolated from the circulatory system. The exceptional architecture of the eye provides ample opportunity for alternative drug delivery methods. In this regard, four non-systemic modes of administration are contemplated by the present invention, namely intravitreal administration (injection), sub-Tenon's injection, iontophoretic delivery, implants/inserts and ophthalmic drop delivery.

The results of ocular irritation and biodistribution studies and inhibition of vessel growth in animal models of corneal, choroidal, or retinal neovascularization, following administration of CA4P are described in the Examples section below.

As such, neovascular retinopathies, as well as ocular tumors, are thus a viable target for CA4P therapy and other tubulin binding agents for a variety of reasons, namely:

Tubulin binding agents may be able to attack the aberrant nascent vessels associated with the retinopathy because these vessels do not share architectural similarities with the BRB. Tubulin binding agents may halt the progression of the disease much like it does with a solid tumor vasculature. In addition, tubulin binding agents may able to cause the regression of nascent vessels as has been observed in various pre-clinical studies.

Since there are no 100%-effective treatments for sub-retinal neovascularization, tubulin binding agents may be effective drugs when used in combination with current state of the art treatments.

Most currently approved treatments for retinopathies involve surgical intervention that may be painful and require long recovery periods. Non-systemic or systemic administration of tubulin binding agents would be a non-surgical form of treatment.

When delivered systemically or nonsystemically, CA4P shows promise as a vascular targeting agent in animal models of corneal, retinal, or choroidal angiogenesis and in animal models with ocular tumors.

As recited, CA4P, as well as other vascular targeting and tubulin binding agents show promise when delivered systemically in models of corneal, retinal, or choroidal angiogenesis, as well as other ocular diseases and tumors. Preferred modes of systemic administration include parenteral and oral administration. Parenteral administration is the route of administration of drugs by injection under or through one or more layers of the skin or mucous membranes. Parenteral routes of administration, by definition, include any route other than the oral-gastrointestinal (enteral) tract. Parenteral administration includes the intravenous, intramuscular and subcutaneous routes.

Pharmaceutical compositions of the invention are formulated to be compatible with its intended route of administration. Pharmaceutical compositions for ophthalmic topical administration may include ophthalmic solutions, ophthalmic gels, sprays, ointments, perfusion and inserts. A topically delivered formulation of tubulin binding agent should remain stable for a period of time long enough to attain the desired therapeutic effects. In addition the agent must penetrate the surface structures of the eye and accumulate in significant quantities at the site of the disease. Additionally, a topically delivered agent should not cause an excessive amount of local toxicity.

Ophthalmic solutions in the form of eye drops generally consist of aqueous media. In order to accommodate wide ranges of drugs which have various degrees of polarity, buffers, organic carriers, inorganic carriers, emulsifiers, wetting agents, etc. can be added. Pharmaceutically acceptable buffers for ophthalmic topical formulations include phosphate, borate, acetate and glucoronate buffers, amongst others. Drug carriers may include water, water mixture of lower alkanols, vegetable oils, polyalkylene glycols, petroleum based jelly, ethylcellulose, ethyl oleate, carboxymethylcellulose, polyvinylpyrrolidone, and isoproplyl myristrate. Ophthalmic sprays generally produce the same results as eye drops and can be formulated in a similar manner. Some ophthalmic drugs have poor penetrability across ocular barriers and are not administrable as drops or spray. Ointments may thus be used to prolong contact time and increase the amount of drug absorbed. Continuous and constant perfusion of the eye with drug solutions can be achieved by placing polyethylene tubing in the conjunctival sac. The flow rate of the perfusate is adjustable via a minipump system to produce continuous irrigation of the eye. Inserts are similar to soft contact lens positioned on the cornea, except that inserts are generally placed in the upper cul-de-sac or, less frequently, in the lower conjunctival sac rather than attached to the open cornea. Inserts are generally made of biologically soluble materials which dissolve in lacrimal fluid or disintegrate while releasing the drug.

In one embodiment, the active compounds are coated upon implants or inserts which are implanted into the eye. One example of such an implant contemplated by the present invention is an implant from Oculex Pharmaceuticals, Inc., Sunnyvale, Calif. The Oculex implant is a biodegradable BDD™ drug delivery device comprised of a biodegradable micro-size polymer system that enables microencapsulated drug therapies to be implanted within the eye. This implant permits the desired drug to be directly released into the area of the eye requiring medication for a predetermined period of time from days, to months to as long as many years.

It is especially advantageous to formulate topical compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Additional known information with regard to the methods for making the formulations in accordance with the present invention can be found in standard references in the field, such as for example, “Remington's Pharmaceutical Sciences”, Mack Publishing Co., Easter, Pa., 15th Ed. (1975).

In addition to the non-systemic routes of administration discussed previously, examples of systemic routes of administration include parenteral, e.g., intravenous, intradermal, subcutaneous, oral (e.g., inhalation), transmucosal, and rectal administration. Solutions or suspensions used for parenteral or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a vascular targeting agent) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In addition to the tubulin binding agents described above, the invention also includes the use of pharmaceutical compositions and formulations comprising a tubulin binding agent in association with a pharmaceutically acceptable carrier, diluent, or excipient, such as for example, but not limited to, water, glucose, lactose, hydroxypropyl methylcellulose, as well as other pharmaceutically acceptable carriers, diluents or excipients generally known in the art.

Another object of the present invention is to provide synergistic combinations of tubulin binding agents and other therapies, such as anti-oxidants, anti-inflammatory compositions such as Interferon Alpha, angiostatic steroids such as Annocortave™, staurosporine derivatives, or antiangiogenic agents that interfere with VEGF-induced neovascularization, such as Angiopoietin-2, Pigment Epithelium-Derived Factor (PEDF), Avastin™, Macugen™. A further object of the present invention is to provide a method of treatment to augment the currently available symptomatic treatments for ocular neovascularization, including mechanical low vision aids, laser photocoagulation therapy, or photodynamic therapy.

As used herein, terms “pharmacologically effective amount”, “pharmaceutically effective dosage” or “therapeutically effective amount” mean that amount of a drug or pharmaceutical agent that will elicit the biological or medical response of a tissue, system, animal or human that is being sought by a researcher or clinician. The appropriate response can include prevention of disease onset, prevention of disease progression, or regression of the disease. In a preferred embodiment, administration of a pharmaceutically effective dosage of the present invention results in regression of subfoveal choroidal neovascularization. A more preferred embodiment results in regression of pathological myopia. Another preferred embodiment results in regression of exudative age-related macular degeneration. As used herein, regression of choroidal neovascularization refers to a a reduction in number of neovascular lesions per retina, a reduction in the average size of neovascular lesions per retina, or a reduction in the total area of neovascularization, as measured by the cumulative size of neovascular lesions in the retina. This decrease in total neovascularization area may be assessed by a variety of techniques, including, for example, fluorescein angiography and image analysis.

The response can be evaluated by visual acuity tests, fluorescein angiograpy, or one of a number of other ocular examinations. A preferred embodiment for evaluating the response is by visual acuity test such that the patient's vision improves by at least two lines on a visual acuity test. In an alternative embodiment, the response can be evaluated by measuring a reduction in the amount of exudate leakage in the treated eye. This decrease in exudates leakage can by measured by a variety of techniques, including, for example, area of hyperfluorescence at different times following fluorescein injection.

The dosage of CA4P for administration to the eye of a subject (non-systemic administration) is in the range of from approximately 0.01 mg/ml to 100 mg/ml. The concentration of CA4P achieved in the eye should be therapeutically relevant and is in the range of approximately 1 nanomolar to 100 millimolar. The more preferred concentration of CA4P in the eye is in the range of from approximately I micromolar to 100 micromolar. When CA4P is administered systemically, an amount of combretastatin A4 prodrug in the range of from approximately 0.1 mg/m² to approximately 120 mg/m² is advantageously administered parenterally. In a particularly preferred embodiment, CA4P is administered intravenously at a dose of 27 mg/m².

It is intended that the systemic and non-systemic administration of tubulin binding agents in accordance with the present invention will be formulated for administration to mammals, particularly humans. However, the invention is not limited in this respect and formulations may be prepared according to veterinary guidelines for administration to animals as well.

The invention is further defined by reference to the following examples. It will be apparent to those skilled in the art that many modifications, both to the materials and methods, may be practiced without departing from the purpose and interest of the invention.

### EXAMPLES

### Example 1

### Ocular Irritation Studies and Determination of Mean Tolerable Dosage (MTD) of CA4P when Administered Locally in the Eye Using Three Routes of Administration

(i) Intravitreal Administration

The test article, CA4P, was evaluated for the potential to cause intraocular irritation following intravitreal injection in rabbits. Following general anesthesia, a 0.2 ml dose of CA4P was administered to the right eyes of eight rabbits. A 0.2 ml dose of 0.9% sodium chloride USP solution was administered to the left eyes of the rabbits to serve as a negative control. Four different concentrations of CA4P were tested. Each of the four concentrations (0.1 mg/ml, 1.0 mg/ml, 10 mg/ml and 100 mg/ml) were dosed to the right eyes of two rabbits. Approximately 48 hours after the treatment, the eyes were examined with a biomicroscopic slit-lamp and an indirect ophthalmoscope. The scores were recorded and the rabbits were euthanized. Immediately after euthanasia, samples of vitreous fluid were drawn and white blood cell counts were determined with a hemacytometer. Counts less than or equal to 200 cells/mm³ were considered to be acceptable.

Control eyes had no significant changes in ocular tissues based on biomicroscopic slit-lamp and ophthalmoscope examination. For test treated eyes, evidence of irritation was noted in one animal at the 1 mg/ml concentration and for both at the 100 mg/ml concentration. Mean cell counts in the vitreous fluid were 9 cells/mm³ for eyes that received the 0.1 mg/ml concentration; 962 cells/mm³ for eyes receiving the 1 mg/ml concentration; 10 cells/mg³ for eyes receiving the 10 mg/ml concentration; 409 cells/mm³ for eyes dosed with the 100 mg/ml concentration, and 5 cells/mm³ for the control eyes.

Under the conditions of the study, the controls reacted as expected with no significant reaction being noted at the ophthalmic examines and vitreal analysis. For the test treated animals, both animals dosed with the 100 mg/ml concentration had pronounced irritation and inflammation at both ophthalmic exams and showed evidence of inflammation from the white blood cell analysis of the vitreous. For all of the lower dose levels (0.1 mg/ml, 1.0 mg/ml and 10 mg/ml), there was no clear evidence of irritation or inflammation

(ii) Sub-Tenon's Administration

The test article, CA4P, was evaluated for primary ocular irritation. Two 0.1 ml injections of the appropriate CA4P concentration (0.1, 1.0, 10 and 100 mg/ml) were injected into the sub-Tenon's space of the right eye of two rabbits. A 0.2 ml portion of buffered saline solution was injected into the sub-Tenon's space of the left eyes to serve as a negative control. Ocular reactions were evaluated at 24, 48 and 72 hours after the sample instillation. On day 3, rabbits were euthanized and eyes were removed. The specimens were fixed and embedded, and histology was performed. Histopathologic changes in the eye tissues were recorded with an emphasis placed on examination of changes in the sub-Tenon's space.

Under the conditions of this study, no irritation was observed in the eyes treated with the 0.1 mg/ml and 1.0 mg/ml concentrations as compared to the corresponding negative control eyes. Slight irritation was observed in the eyes treated with 10 mg/ml and 100 mg/ml concentrations as compared to the corresponding negative control eyes.

(iii) Topical Drops

The test article, CA4P, was evaluated for primary ocular irritation. A single 0.2 ml dose of CA4P dilution (0.1, 1.0, 10 and 100 mg/ml) was placed in the lower conjunctival sac of the left eye to serve as a comparative control. The contralateral eye received buffered saline solution. Ocular reactions were evaluated at 24, 48 and 72 hours after the sample instillation.

Under the conditions of this study, the macroscopic reaction of all test article dilutions was considered insignificant as compared to that of the control. Microscopically, the test article was not considered an irritant as compared to the buffered saline solution control article.

A summary of the results are set forth in the table:

Draize Scores

| | |
|---|---|
| Administration | Irritation* |

| | | | |
|---|---|---|---|
| Topical | 0-10 | mg | No Irritation |
| Intravitreal | <1 | mg | No Irritation |
| | 10 | mg | Irritation |
| Sub-Tenon's | 0-10 | mg | No Irritation |

*Dose range: 0.001, 0.01, 0.1, 1.0, 10 mg

### Example 2

### Assessment of the Biodistribution of CA4P when Administered Locally in the Eye Using Different Routes of Administration

To be effective in the treatment of ocular neovascularization, a non-systemic method of drug administration must penetrate the relevant structures of the eye and deliver the drug in therapeutically significant quantities at the disease site. To confirm that the various methods of non-systemic injection would result in significant biodistribution of CA4P, radiolabelled drug biodistribution experiments were performed.

Methods:

Unless noted below, the following experimental protocol was followed for each biodistribution experiment.

¹⁴C-CA4P (OXiGENE Inc., Watertown, Mass.) was resuspended in 100 ul volume of saline solution and injected with a 30 G needle into the right eyes of anesthesized male New Zealand rabbits (4 months old, 1.8-2.5 kg, n=3 per sample). Three different concentrations of ¹⁴C-CA4P were tested (1, 10, 100 mg/ml) corresponding to doses of 1, 5, and 5 uCi of applied radioactivity respectively. A blank control group was also included. Rabbits were anaesthetized at 1, 6, 24, and 48 hours for blood sampling. After blood collection, the animals were euthanized by Phenobarbital injection and the treated right eyes were removed from all test animals. Ocular tissue samples were dissected from the cornea, aqueous humor, vitreous humor, choroid, or retina, placed in 20-mi glass scintillation vials, vortexed, and incubated for 24 hrs with 500 ul digesting fluid. Plasma was separated from whole blood by centrifugation (1,800 g for 10 minutes). Both ocular tissue samples and plasma were incubated at room temperature with 16 ml of Hionic Fluor™ scintillation fluid for a period of 24 hours prior to radioactivity counting. Each sample was counted for 5 minutes in a Betamatic V counter (Bio-Tek Kontron Instruments, St Quentin en Yvelines, France). The conversion of counts per minute (“cpm”) into disintegrations per minute (“dpm”) was performed automatically by the beta-counter, using calibration curves obtained from ¹⁴C-standards and quenching curves from the respective blank matrices spiked with ¹⁴C-standards. The concentration of drug was determined according to nanogram equivalents of CA4P (ng-Eq/g of tissue) which was calculated from the measured dpm value, the weight of the tissue specimen, and the specific activity of the drug (0.37 mCi/mg), followed by subtraction of the corresponding background value from control eye tissue. A tissue concentration of 1 uM CA4P is equal to 440 nEq/g tissue.

Results:

(i) Intravitreal Administration

Table 1 recites the biodistribution results following intravitreal injection. In all tissues examined, the degree of ocular penetration was dependent on the concentration of CA4P placed on the surface of the eye. The highest concentrations of drug in the eye (“Cₘₐₓ”) were achieved within the first hour following administration. Therapeutically relevant concentrations of drug (>1 uM) were delivered to the retina at all concentrations tested. High concentrations of drug were also found in the vitreous and sclera. Relatively little drug was found in the aqueous humor of the eye or the blood plasma.

TABLE 1
Biodistribution of CA4P Following Intravitreal Injection

| Ocular Tissue | Dose | |
|---|---|---|
| Sample | (mg/ml) | Cₘₐₓ (uM) |
| Aqueous Humor | 100 | 460 |
| | 10 | 0.79 |
| | 1 | 0.15 |
| Vitreous | 100 | 10,039 |
| | 10 | 757 |
| | 1 | 75 |
| Retina | 100 | 10,696 |
| | 10 | 1,981 |
| | 1 | 160 |
| Cornea | 100 | 5,871 |
| | 10 | 969 |
| | 1 | 95 |
| Sclera | 100 | 16,112 |
| | 10 | 153 |
| | 1 | 19 |
| Plasma | 100 | 0.78 |
| | 10 | 0.2 |
| | 1 | 0.4 |

(ii) Sub-Tenon's Administration

(1) Biodistribution

Table 2 recites the biodistribution results following Sub-Tenon's injection. In all tissues examined, the degree of ocular penetration was dependent on the concentration of CA4P placed on the surface of the eye. The highest concentrations of drug in the eye were within the first hour following administration. Therapeutically relevant concentrations of drug (>1 uM) were delivered to the retina and choroid at the 100 and 10 mg/ml administered dose. A high concentration of drug was also observed in the sclera. Relatively little drug was found in the vitreous, aqueous humor, or the blood plasma.

TABLE 2
Biodistribution of CA4P Following Sub-Tenon's Injection

| Ocular Tissue | | |
|---|---|---|
| Sample | Dose (mg/ml) | Cₘₐₓ (uM) |
| Aqueous | 100 | 5.92 |
| Humor | 10 | 1.8 |
| | 1 | 0.3 |
| Vitreous | 100 | 3.9 |
| | 10 | 0.13 |
| | 1 | 0.019 |
| Retina | 100 | 171 |
| | 10 | 7.1 |
| | 1 | 0.85 |
| Choroid | 100 | 861 |
| | 10 | 6.2 |
| | 1 | 1.2 |
| Plasma | 100 | 9.3 |
| | 10 | 0.49 |
| | 1 | 0.07 |

(iii) Subconjunctival Administration

Subconjuctival injections were administered at doses of 0.1, 1, 10, and 100 mg/ml. ¹⁴C-CA4P solutions were formulated with 0.24% 10N KOH and 0.01% Benzalkonium chloride and injected in a volume of 100 ul. Animals were treated at an applied dose of 5 uCi with a single subconjunctival injection in right eyes. After delivery, the eye was gently held closed for 2-5 seconds. The results of the experiment are recited in Table 3 below.

The highest concentrations of drug in the eye were within the first hour following administration. Therapeutically relevant concentrations of drug (>1 uM) were delivered to the cornea, retina, and choroid at the 100, 10, and 1 mg/ml administered does. Relatively little drug was found in the vitreous or the blood plasma.

TABLE 3
Biodistribution of CA4P Following Subconjunctival Injection

| Ocular Tissue | | |
|---|---|---|
| Sample | Dose (mg/ml) | Cₘₐₓ (uM) |
| Cornea | 100 | 761 |
| | 10 | 143 |
| | 1 | 13 |
| | 0.1 | 1.5 |
| Vitreous | 100 | 15.7 |
| | 10 | 2.1 |
| | 1 | 0.02 |
| | 0.1 | 0.005 |
| Retina | 100 | 174 |
| | 10 | 39.6 |
| | 1 | 1.7 |
| | 0.1 | 0.13 |
| Choroid | 100 | 908 |
| | 10 | 188 |
| | 1 | 1.9 |
| | 0.1 | 2.3 |
| Plasma | 100 | 8 |
| | 10 | 0.9 |
| | 1 | 0.025 |
| | 0.1 | 0.017 |

(iv) Periocular Administration

Table 4 recites the biodistribution results following Periocular injection. In all tissues examined, the degree of ocular penetration was dependent on the concentration of CA4P placed on the surface of the eye. The highest concentrations of drug in the eye were within the first hour following administration. Therapeutically relevant concentration of drug (>1 uM) were delivered to the retina and choroid at all administered doses. A high concentration of drug was also observed in the sclera. Relatively little drug was found in the vitreous or the blood plasma.

TABLE 4
Biodistribution of CA4P Following Periocular Injection

| Ocular Tissue | | |
|---|---|---|
| Sample | Dose (mg/ml) | Cₘₐₓ (uM) |
| Vitreous | 100 | 3.35 |
| | 10 | 0.34 |
| | 1 | 0.03 |
| Retina | 100 | 169 |
| | 10 | 54 |
| | 1 | 4.4 |
| Choroid | 100 | 1,040 |
| | 10 | 74.5 |
| | 1 | 14.3 |
| Sclera | 100 | 3,366 |
| | 10 | 280 |
| | 1 | 18 |
| Plasma | 100 | 12.3 |
| | 10 | 0.79 |
| | 1 | 0.07 |

(v) Topical Formulations

Topical gels and solutions were developed for use as topical formulation suitable for the topical delivery of CA4P to the surface of the eye. Topical solutions (1, 3, and 10%) was directly prepared in 0.9% NaCl (Aguettant, Lyon, France) and sterilized with 0.2 um filter (pH 6.4 to 8.5, osmolarity 290 to 459 mosmol/kg H2O. Low viscosity topical gels (1,3,and 10%) were prepared in 0.5% carboxymethylcellulose (Sigma Aldrich Chimie, St. Quentin Fallavier Cedex, France) with 0.9% NaCl. The physicochemical specifications of each gel are listed in Table 5

TABLE 5
Topical CA4P Gel Formulations.

| | 1% CA4P Gel | | 3% CA4P Gel | | 10% CA4P Gel | |
|---|---|---|---|---|---|---|
| Test | Spec | Result | Spec | Result | Spec | Result |
| pH | 7.4-8.1 | 7.796 | 7.7-8.4 | 8.203 | 8.1-8.8 | 8.491 |
| Osmolarity | 330-370 | 354 | 290-330 | 315 | 475-515 | 495 |
| Viscosity | 30-80 | 49 | 30-80 | 58 | 50-100 | 71 |
| (mPa · s) | | | | | | |

Topical formulations were applied to the surface of right eyes at an applied dose of 5 uCi in a volume of 50 ul. Cornea was sampled instead of sclera. Samples were taken at 0.5, 1.6 and 24 hours.

Table 6 recites the biodistribution results following administration of each topical CA4P gel formulation. In all tissues examined, the degree of ocular penetration was dependent on the concentration of CA4P in each gel formulation. The highest concentrations of drug in the eye were within the first hour following administration. Therapeutically relevant concentrations of drug (>1 uM) were delivered to the cornea, retina, and choroid with all three gel formulations. Relatively little drug was found in in the blood plasma.

TABLE 6
Biodistribution of CA4P following Topical
Administration of a Gel

| Ocular Tissue | | |
|---|---|---|
| Sample | Dose (% CA4) | Cₘₐₓ (uM) |
| Cornea | 10 | 292 |
| | 3 | 118 |
| | 1 | 82 |
| Aqueous | 10 | 13 |
| Humor | 3 | 8.2 |
| | 1 | 2.4 |
| Choroid | 10 | 22.5 |
| | 3 | 10.7 |
| | 1 | 2.8 |
| Retina | 10 | 5.8 |
| | 3 | 5.8 |
| | 1 | 1.0 |
| Plasma | 10 | 1.63 |
| | 3 | 0.56 |
| | 1 | 0.09 |

Table 7 recites the biodistribution results following administration of each topical CA4P solution formulation. In all tissues examined, the degree of ocular penetration was dependent on the concentration of CA4P in each solution formulation. The highest concentrations of drug in the eye were within the first hour following administration. Therapeutically relevant concentrations of drug (>1 uM) were delivered to the cornea with all three solution formulations, while 10 mg/ml dose resulted in delivery of a significant amount of drug to the retina and choroid.

TABLE 7
Biodistribution of CA4P following Topical Administration
of a Solution Formulation

| Ocular Tissue | | |
|---|---|---|
| Sample | Dose (% CA4) | Cₘₐₓ (uM) |
| Cornea | 10 | 104 |
| | 3 | 34 |
| | 1 | 10 |
| Aqueous | 10 | 4.1 |
| Humor | 3 | 1.9 |
| | 1 | 0.6 |
| Choroid | 10 | 15.6 |
| | 3 | 1.9 |
| | 1 | 0.87 |
| Retina | 10 | 4.2 |
| | 3 | 0.39 |
| | 1 | 0.27 |
| Plasma | 10 | 1.27 |
| | 3 | 0.84 |
| | 1 | 0.07 |

It is apparent from these experiments that non-systemic delivery of CA4P by any of a variety of methods is effective in achieving a therapeutically relevant concentration of drug in the cornea, retina, or choroids. Each of these tissues is a potential site of ocular neovascularization.

### Example 3

### Ocular Administration of CA4P via Iontophoresis

CA4P is ionizable at physiological pH and therefore is amenable to iontophoretic delivery. The effectiveness of transcleral iontophoretic delivery of CA4P was evaluated using an ocular rabbit ophthalmic applicator (IOMED Inc.,.Salt Lake City, Utah) composed of an 180 ul silicone receptacle shell backed with silver chloride-coated silver foil current distribution component, a connector lead wire, and a single layer of hydrogel-impregnated polyvinyl acetal matrix to which CA4P (10 mg/ml) was administered. The contact surface area of the applicator was 0.54 cm². The applicator was placed over the sclera in the right eyes of New Zealand white rabbits (3-3.5 kg, n=6 for each treatment) in the superior cul-de-sac at the limbus with the front edge 1-2 mm distal from the corneoscleral junction. Direct current anodal iontophoresis was performed with each applicator at 2,3,and 4 mA for 20 min using an Phoresor II™ PM 700 (IOMED Inc., Salt Lake City, Utah) power supply. Passive iontophoresis (0 mA for 20 min) was used as a control. Following treatment, the animals were euthanized, and eyes were enucleated 30 minutes post-treatment, rinsed with tap water, and frozen at −70 C. Retina and choirodal tissue was dissected from these sample.

CA4P, CA, and the internal standard Diethylstilbestrol (Sigma Chemical Company) were quantified from approximately 100 mg of tissue using chromatography tandem mass spectrometry (“LC/MS/MS”) method. An aliquot of methonal extraction was injected onto a SCIEX APIO 3000 LC/MS/MS apparatus equipped with an HPLC colum. Peak area of the m/z 315→285 product ion of CA43 and m/z 395→79 product ion of CA4P were measured against the peak area of the m/z 267→237 product ion of the internal standard. Quantitation was performed using weighted (1/x) linear least squares regression analyses generated from fortified calibration standards prepared immediately prior to each run. The initial combretastatin amounts were significantly higher than the quantification range, therefore had to be extrapolated after analysis. As Table 8 demonstrates, the delivery of total combretastatin to the choroids and retina is enhanced approximately 15-fold by iontophoresis when compared to passive delivery. These levels represent a several thousand-fold excess over what is considered to be a therapeutically relevant concentration for inhibiting tubulin binding (2-3 uM). There did not appear, however, to be a current-dependence of delivery to the retina/choroid.

TABLE 8
Iontophoretic Enhancement of Combretastatin Delivery to
Retina/Choroid. (Mean ± SD)

| | | | Total | |
|---|---|---|---|---|
| | | | Com- | |
| | | | bretastatin | |
| | Amount CA4P | Amount CA4 | Delivered | En- |
| Treatment | Delivered (ng) | Delivered (ng) | (nmol/g) | hancement |
| 0 mA, 20 min | <0.4 | 57 ± 37 | 1.6 ± 1.0 | NA |
| 2 mA, 20 min | 1.4 ± 0.5 | 910 ± 630 | 27 ± 15 | 17 |
| 3 mA, 20 min | 3.8 ± 1.5 | 710 ± 450 | 25 ± 17 | 16 |
| 4 mA, 20 min | 7.2 ± 6.3 | 670 ± 440 | 24 ± 10 | 15 |

### Example 4

### Treatment of Corneal Neovascularization via Systemic Administration of CA4P

To simulate pathogenic ocular angiogenesis, ocular neovascularization was induced by administration of lipid hydroperoxide (LHP) by intra-corneal injection at a dosage of 30 μg to rabbit eyes. Seven to 14 days later, ocular vessels formed in the injected eyes due to LHP insult. The subjects were divided into two groups; those of one group were given combretastatin A4 disodium phosphate by intravenous administration at a dosage of 40 mg/kg once a day for five days, while a vehicle without combretastatin A4 disodium phosphate was administered to the other group by i.v. administration as a dosage of water for the same time period. The eyes of both groups were examined seven days later. A reduction of vessels of 40% or more was observed in the group treated with combretastatin A4 disodium phosphate, but not in the other group.

### Example 5

### Treatment of Corneal Neovascularization via Systemic Administration of CA4P

To assess the ability of CA4P to inhibit corneal neovascularization, a rabbit corneal model was used in which neovascularization was induced by linoleic acid hydroperoxide (“LHP”) injection (Ueda et al., Angiogenesis, 1997, 1: 174-184). Injection of LHP in the corneal stroma stimulates the localized production of angiogenic cytokines within the cornea. Blood vessels in the circumlimbal plexus respond to the angiogenic stimulation by migrating towards the site of LHP injection. Therapeutic efficacy of systemically delivered CA4P was assessed by measuring the length of these proliferating vessels.

Experimental Methods:

As outlined in Table 9 below, adult male New Zealand rabbits (2.7-3.0 kg) were injected with 10 ul suspension of LHP (60 ug) 5 mm from the superior limbus to induce corneal angiogenesis. Vessels grew at a rate of 0.25 mm/day. Groups 2 and 4 were injected intraperitoneally (“IP”) with CA4P (50 mg/kg) after 3 and 10 days of vessel growth respectively. Treatment groups 1 and 3 were injected with saline control on day 3 and day 10 post-LHP injection. Surface photographs of the cornea were taken at 0, 3, 6, 12, 17, and 28 days post-LHP injection. Following each photographic session, corneal vessels were observed under an operating microscope and Castroviejo calipers were used to measure the length of the most prominent vessel.

In addition to longest-vessel measurements, histology analysis was undertaken on day 28 to assess the amount of dissolved extracellular matrix, vessel wall thickness, and degree of vessel branching. Euthanized animals were enucleated and the vitreous was removed from each eye prior to fixation in 4% paraformaldehyde for 45 minutes and 0.2M cacodylate buffer (pH 7.4) overnight. Eyes were embedded in paraffin, sectioned to a 3 um thickness, and stained with Hemolysin and eosin.

TABLE 9
Experimental Design

| | Sample | | | | Sacrifice |
|---|---|---|---|---|---|
| Group | Size | Treatment | Day | Schedule | Day |
| 1 | n = 4 | Vehicle | 3 | qd × 5 | 28 |
| | | (PBS) | | | |
| 2 | n = 5 | CA4P | 3 | qd × 5 | 28 |
| | | (50 mg/kg) | | | |
| 3 | n = 2 | Vehicle | 10 | qd × 5 | 28 |
| | | (PBS) | | 2 day rest | |
| | | | | qd × 5 | |
| 4 | n = 7 | CA4P | 10 | qd × 5 | 28 |
| | | (50 mg/kg) | | 2 day rest | |
| | | | | qd × 5 | |

Results:

Table 10 and 11 summarize the effects of CA4P on vessel length as a function of intervention-time and number of treatments. When CA4P treatment was used to intervene within 3 days of the initial angiogenic stimulation (Table 10, Group 2), the drug caused a complete inhibition of neovascular growth. In contrast, vessels in the vehicle control group continued to grow. This effect can be qualified as angiogenesis inhibition or an anti-angiogenic effect. When CA4P treatment was used to intervene 10 days after the angiogenic stimulation (Table I 1, Group 2), the effect was the same.

TABLE 10
Early Intervention: Treatment begins on Day 3

| | Vessel Length | Vessel Length | Vessel Length |
|---|---|---|---|
| Group | (mm) on Day 3 | (mm) on Day 6 | (mm) on Day 12 |
| Vehicle | 0.8 ± 0.12 | 1.9 ± 0.04 | 3.7 ± 0.13 |
| Control | | | |
| (Group 1) | | | |
| 50 mg/kg | 0.6 ± 0.12 | 0.7 ± 0.16 | 0.5 ± 0.61 |
| CA4P | | | |
| (Group 2) | | | |
| P Value | | >0.001 | >0.001 |

TABLE 11
Late Intervention: Treatment begins on Day 10

| | Vessel Length | Vessel Length | Vessel Length |
|---|---|---|---|
| Group | (mm) on Day 12 | (mm) on Day 12 | (mm) on Day 24 |
| Vehicle | 4.0 ± 0.7 | 5.4 ± 0.6 | 6.4 ± 0.3 |
| Control | | | |
| (Group 3) | | | |
| 50 mg/kg | 2.6 ± 0.29 | 2.8 ± 0.49 | 1.4 ± 0.46 |
| CA4P | | | |
| (Group 4) | | | |
| P Value | | >0.05 | >0.05 |

FIG. 3B is a surface photograph of a CA4P-treated eye on Day 28. This photograph further illustrates the inhibition of vessel growth on Day 28 following CA4P administration in comparison with the vehicle control eye depicted in FIG. 3A.

The micrographs presented in FIGS. 4A and 4B (magnification 400×) are examples of the stained histological specimens obtained from the same animals on day 28. In the vehicle-treated animals (FIG. 4A), vessels appeared round and numerous. In contrast, in CA4P treated animals (FIG. 4B) vessels appeared narrow and less numerous. In addition, evidence of vessel regression was observed at during later stages of intervention with CA4P (data not shown). It appeared that CA4P was able to reduce the width of the established vessels and significantly inhibit the sprouting of branches from thee vessels, which is indicative of an additional vascular targeting effect.

### Example 6

### Treatment of Choroidal Neovascularization in an Animal Model of Macular Degeneration via Systemic Administration of CA4P

Choroidal neovascularization is a major cause of severe vision loss in patients with age-related or wet macular degeneration. To investigate the capacity of CA4P to inhibit vascular growth in the choroids, a murine model of Choroidal Neovascularization was tested. In this model the investigator used a krypton laser to create a wound on the Bruch's membrane of a C57BL/6J mouse. Each eye received several burns. The burn elicited a classic wound-healing response that included neovascularization within the choroid. This krypton laser photocoagulation method has been described in Tobe et al., Am. J. of Pathology, 1998, 153(5):1641-6. In a subset of animals (n=19), CA4P was systemically administered by IP injection at a dose of 100 mg/kg/day. Histopathology and fluorescein angiography were used to identify neovascularization surrounding the burn. Electron microscopy was used to measure the lumen diameter of fenestrated neovasculature within the choroidal neovascular lesions. Table 12 illustrates the results of CA4P treatment on the average vessel lumen area. Animals treated with CA4P possessed approximately 50 % less vascular lumen area (mm²) when compared to animals treated with saline (n=33). Statistical analysis of the results demonstrated a high degree of significance.

TABLE 12
Average Lumen area of CA4P-treated and vehicle-mice

| | CA4P | CA4P | CA4P | |
|---|---|---|---|---|
| MOUSE | (100 mg/kg) | (20 mg/kg) | (10 mg/kg) | Vehicle |
| # Mice in | 19 | 11 | 10 | 33 |
| Group | | | | |
| Average Vessel | 0.0076115 | 0.0110057 | 0.011858 | 0.0129886 |
| Lumen Area | | | | |
| (mm²) | | | | |
| Standard | 0.0032669 | 0.0043229 | 0.0041457 | 0.0047336 |
| Deviation | | | | |

### Example 7

### Treatment of Retinal Neovascularization in a Mouse Model of Retinopathy of Prematurity via Systemic Administration of CA4P

The inner retina of the mammalian eye receives oxygen from the superficial retinal capillary bed. This capillary bed is located beneath the inner limiting membrane which serves as the interface between the inner retina and the outer avascular vitreous. The pathology of retinal neovascularization or retinopathy arises from ischemia-induced growth of neovasculature beyond the retinal inner limiting membrane and into the vitreous, causing severe loss of vision and frequently leading to retinal detachment. A well-characterized murine model of oxygen-induced retinal neovascularization closely simulates retinopathy of prematurity (“ROP”) exhibited by prematurely born human infants, and exhibits characteristics common to a variety of other ischemia-induced retinopathies, including diabetic retinopathy (Smith et al., Invest. Ophthalmol. Vis. Sci., 1994, 35:101-11). In this model neonatal mice are exposed to sustained hyperoxic conditions (75% oxygen for 7 days) that inhibit the development of the superficial retinal capillary bed. When a mouse pup is removed from the pure oxygen environment and placed in the relative hypoxia of environmental oxygen, the underdeveloped superficial retinal capillary bed is unable to deliver sufficient quantities of oxygen to the retina. The retina responds to the lack of oxygen by producing angiogenic cytokines that cause serious pathological consequences. The localized production of angiogenic cytokines can cause the underdeveloped superficial retinal capillary bed to sprout new vessels that breach the inner limiting membrane. The growth of the aberrant blood vessels in the vitreous causes the formation of severe scar tissues and traction-induced retinal detachment.

It is expected that the treatment of a neonatal mouse with CA4P immediately upon its removal from hyperoxic conditions would be an effective treatment method for ROP. Retinal neovascularization can be quantified by counting the number chemically stained nuclei of penetrating endothelial cells in retinal tissue section of treated and untreated eyes according to existing methods (Majka et al., Invest. Ophthalmol. Vis. Sci. 2001, 42: 210-15). It is expected that the number of nuclei penetrating the inner limiting membrane would be significantly reduced in CA4P eyes.

### Example 8

### Treatment of Ocular Tumors in a Mouse Model of Retinoblastoma via Subconjuctival Administration of CA4P

A murine transgenic model of retinoblastoma was used in which SV-40 Large T antigen positive mice develop bilateral retinoblastoma resembling human pediatric retinoblastoma. In this model, tumors first appear at 4 weeks of age and develop in a stable and reproducible manner (Hayden et al., Arch Ophthalmol. 2002;120(3):353-9). In one experiment, 12-week old animals (n=48) were treated with a single 20 ul subconjunctival injection of CA4P (100 mg/ml) in the right eye only. Control mice (n=8) were treated with a balanced salt solution (“BSS”). Eyes were sampled by enucleation at Days 1, 3, 7, 14, 21, and 28 post treatment (n=8 eyes per sample). Samples were fixed, paraffin embedded, serially sectioned, and stained with heometoxylin, eosin, and PAS prior. Each tissue sample was examined for histopathological tumor vascular response. As illustrated in FIG. 5A, a significant reduction in intratumoral vascularization was apparent on Days 1, 3, and 7.

In a separate dosing experiment, animals (n=48) were treated with 6 serial biweekly subconjunctival injections of CA4P, at concentrations of 100, 10, 1, 0.1 or 0.01 mg/ml in a volume of 20 ul (n=6 eyes per sample). A control group (n=6) received serial subconjunctival injections of BSS. Eyes were enucleated at 28 days post-treatment and examined for tumor volume reduction. FIG. 5B illustrates the dose-dependent effect of CA4P on tumor vascular volume in comparison to control. No intratumoral vascularity was present at treatment dose levels above 10 mg/ml and no evidence of toxicity was noted at any time point or treatment dose.

### Example 9

### Treatment of Pathological Myopia in a Human Patient via Systemic Administration of CA4P

A 35 year old male was originally examined on Day One after complaining of visual obstructions in his left eye. The patient had received ocular lens implants in both eyes approximately 2 years earlier to correct for myopia. The patient was diagnosed with pathological myopia (also known as proliferative myopathy and myopic macular degeneration) and received a total of four treatments of Photodynamic Therapy (PDT, Visudyne™) in the left eye over the course of the next 8 months. However, the patient again complained of severe vision loss in the left eye and upon examination the left eye exhibited active leakage of blood and fluid. In June 2003, the patient was diagnosed with pathological myopia in the right eye as well.

About 3 months later, the patient was enrolled in an open-label, pilot (phase I/II), dose-escalation safety and tolerability study of CA4P. At that time, the patient's best corrected visual acuity was 20/50-3 in the left eye and 20/25-3 in the right eye, as determined by a Snellen back-lit visual acuity test. Upon examination both eyes exhibited active leakage of blood and fluid. On Day One of the study, the patient began treatment with an intravenous infusion of CA4P (free acid), at a dose of 27 mg/m2, over a 10 minute period. On Day 8 of the study, the the patient exhibited a visual acuity of 20/20-1 in the left eye and 20/20-0 in the right eye. No active leakage was observed in FA of either eye. The patient received second, third, and fourth infusions of CA4P on Days 8, 15, and 22 of the study, with a maintenance of visual acuity and no active leakage in either eye. The patient subjectively reported that his vision had improved dramatically since beginning CA4P treatment, and notably reported that he could read text of normal font size.

The finding from the case history example given above are as follows:

1. Subjective Visual Improvement. The patient reported that his deteriorating vision had improved remarkably since beginning treatment with CA4P.

2. Objective Visual Improvement. The patient's Snellen visual acuity had improved by five lines to 20/20 vision in the left eye.

3. FA assessment of Pathological Myopia. Before treatment with CA4P was begun, evaluation of both of the patient's eyes revealed fluid leakage and bleeding. By comparison, no bleeding or exudate was observed in FA was observed immediately following treatment and for the remainder of the study.

### Example 10

### Treatment of Age-Related Macular Degeneration in Human Patients via Systemic Administration of CA4P.

Three patients, aged 50 years or older, were enrolled in an open-label, pilot (phase I/II), dose-escalation safety and tolerability study of CA4P. Each patient met the study entry criteria of less than 20/40 visual acuity in the study eye and better or equal to 20/800 visual acuity in the fellow eye, as determined by Early Treatment Diabetic Retinopathy Study (ETDRS). Fluorescein Angiography (FA) of each patient's study eye revealed subfoveal choroidal neovascularization secondary to age-related macular degeneration, with a total lesion size (including blood, atrophy/fibrosis, and neovascularization) of less than 12 total disc areas, of which at least 50% were comprised of active choroidal neovascularization. None of the patients exhibited clinically significant cardiac abnormalities or evidence of QTc prolongation. In addition, none of the patients had previously received subfoveal thermal laser therapy or any other ocular treatment within 12 weeks prio to screening. None of the patients more than 25% scarring or atrophy, and all had clear ocular media and adequate papillary dilatation to permit good quality stereoscopic fundus photography.

On Day One of the study, following completion of a 2-4 week evaluation period, each patient began treatment with an intravenous infusion of 27 mg/m2 dose CA4P free acid in saline solution, administered over a 10 minute period. Each patient received a second, third, and fourth infusion of CA4P (27 mg/m2 dose free acid solution) on Day 8, Day 15, and Day 22 of the study. An infusion pump or syringe pump coupled with an in-line filter (<5 microns) was used for the administration of CA4P. CA4P for Injection consisted of a sterile freeze-dried, disodium salt, with sufficient excess in the vial to provide 90 mg of the free acid. Each vial of CA4P for Injection was constituted with 11 ml sterile water for injection, USP, to yield a concentration of 9 mg/ml of drug product as the free acid. This was further diluted with approximately 100 ml to 150 ml normal saline to achieve conventrations between 0.6 mg/ml and 1.1 mg/ml, as the free acid, prior to IV administration. The total dose of CA4P that is administered was rounded to the nearest milligram and Body Surface Area was calculated using the actual height and weight of the patient. For patient with a BSA>2.0 m2, the CA4P dose was calculated using a BSA=2.0 m2.

FA was performed 1 hour following the first infusion, and immediately before the second, third, and fourth infusions of CA4P. FA was also performed during follow-up examination at 4 weeks and 8 weeks following the fourth infusion of CA4P. The amount of exudate leakage was measured by the difference in the area of hyperfluorescence 30 seconds and 3 minutes after fluorescein injection. The area of neovascularization was scored using templates superimposed on projected images of the FA. Lesion composition and retinal thickness was also assessed by Optical Coherence Tomography (OCT) during each ocular examination. The visual acuity and of each patient was also assessed during each ocular examination by ETDRS protocol refraction.

Patients have exhibited 2 or 3 lines of improvement in visual acuity tests.

### Other Embodiments

It is to be understood that while the invention has been described in conjunction with the detailed description thereof, the foregoing description is intended to illustrate and not limit the scope of the invention, which is defined by the scope of the appended claims.

It is also to be understood that the drawings are not necessarily drawn to scale, but that they are merely conceptual in nature.

What is claimed is:

1. A method for the treatment or prevention of choroidal neovascularization, the method comprising the steps of:
a) preparing a dosage comprising a pharmaceutically effective dosage of a tubulin binding agent;
b) administering the pharmaceutically effective dosage to a subject in need thereof.

2. The method as recited in claim 1, wherein said tubulin binding agent is combretastatin A4.

3. The method as recited in claim 1, wherein said tubulin binding agent is combretastatin A4 prodrug.

4. The method as recited in claim 1, wherein said choroidal neovascularization is subfoveal.

5. The method as recited in claim 1, wherein said choroidal neovascularization is present in a subject suffering from exudative age related macular degeneration or pathological myopia.

6. The method as recited in claim 1, wherein said pharmaceutically effective dosage is administered systemically to the eye of said subject.

7. The method as recited in claim 1, wherein said pharmaceutically effective dosage is administered by intravenous infusion.

8. The method as recited in claim 6, wherein said pharmaceutically effective dosage administered systemically comprises an amount of combretastatin A4 prodrug in the range of from approximately 0.1 mg/m² to approximately 120 mg/m².

9. The method as recited in claim 6, wherein said pharmaceutically effective dosage administered systemically comprises an amount of combretastatin A4 prodrug in the range of from approximately 2 mg/m² to approximately 90 mg/m².

10. The method as recited in claim 6, wherein said pharmaceutically effective dosage administered systemically comprises an amount of combretastatin A4 prodrug in the range of from approximately 15 mg/m² to approximately 50 mg/m².

11. The method as recited in claim 6, wherein said pharmaceutically effective dosage administered systemically comprises approximately 27 mg/ m² of the free acid of combretastatin A4 phosphate.

12. The method as recited in claim 11, wherein said pharmaceutically effective dosage is administered once a week for four weeks.

13. A method for improving visual acuity in a subject suffering from choroidal neovascularization which comprises periodically administering a dosage of tubulin binding agent to said subject.

14. The method as recited in claim 13, wherein said subject exhibits improvement of at least two lines in a visual acuity test.

15. The method as recited in claim 13, wherein said tubulin binding agent is combretastatin A4.

16. The method as recited in claim 13, wherein said tubulin binding agent is the free acid of combretastatin A4 phosphate.

17. The method as recited in claim 16, said dosage is in the range of from approximately 2 mg/m² to approximately 90 mg/m².

18. The method as recited in claim 16, wherein said dosage is in the range of from approximately 15 mg/m² to approximately 50 mg/m².

19. The method as recited in claim 16, wherein said dosage comprises approximately 27 mg/ m².

20. The method as recited in claim 19, wherein said pharmaceutically effective dosage is administered once a week for four weeks.

21. A method to reduce the leakage of exudate from a lesion in the eye of a subject having choroidal neovascularization and identified as having a lesion, said method comprising periodically administering a dosage of tubulin binding agent to said subject.

22. The method as recited in claim 21, wherein said tubulin binding agent is combretastatin A4.

23. The method as recited in claim 21, wherein said tubulin binding agent is the free acid of combretastatin A4 phosphate.

24. The method as recited in claim 23, said dosage is in the range of from approximately 2 mg/m² to approximately 90 mg/m².

25. The method as recited in claim 23, wherein said dosage is in the range of from approximately 15 mg/m² to approximately 50 mg/m².

26. The method as recited in claim 23, wherein said dosage comprises approximately 27 mg/ m².

27. The method as recited in claim 26, wherein said pharmaceutically effective dosage is administered once a week for four weeks.

28. A method for inducing regression of proliferating vasculature in the eye of a subject suffering from choroidal neovascularization, said method comprising periodically administering a dosage of tubulin binding agent to said subject.

29. The method as recited in claim 28, wherein said tubulin binding agent is combretastatin A4.

30. The method as recited in claim 28, wherein said tubulin binding agent is the free acid of combretastatin A4 phosphate.

31. The method as recited in claim 30, said dosage is in the range of from approximately 2 mg/m² to approximately 90 mg/m².

32. The method as recited in claim 30, wherein said dosage is in the range of from approximately 15 mg/m² to approximately 50 mg/m².

33. The method as recited in claim 30, wherein said dosage comprises approximately 27 mg/m².

34. The method as recited in claim 33, wherein said pharmaceutically effective dosage is administered once a week for four weeks.

35. A method for suppressing the growth of proliferating vasculature in the eye of a subject suffering from choroidal neovascularization, said method comprising periodically administering a dosage of tubulin binding agent to said subject.

36. The method as recited in claim 35, wherein said tubulin binding agent is combretastatin A4.

37. The method as recited in claim 35, wherein said tubulin binding agent is the free acid of combretastatin A4 phosphate.

38. The method as recited in claim 37, said dosage is in the range of from approximately 2 mg/m² to approximately 90 mg/m².

39. The method as recited in claim 37, wherein said dosage is in the range of from approximately 15 mg/m² to approximately 50 mg/m².

40. The method as recited in claim 37, wherein said dosage comprises approximately 27 mg/ m².

41. The method as recited in claim 40, wherein said pharmaceutically effective dosage is administered once a week for four weeks.

42. A pharmaceutical composition for the treatment or prevention of choroidal neovascularization which comprises approximately 15 mg/m² to approximately 50 mg/m² of the free acid of combretastatin A4 phosphate together with a pharmaceutically acceptable carrier, excipient, diluent or adjuvant for systemic administration to a subject in need thereof.

43. A pharmaceutical composition for the treatment or prevention of choroidal neovascularization which comprises approximately 15 mg/m² to approximately 50 mg/m² of the free acid of combretastatin A4 phosphate together with a pharmaceutically acceptable carrier, excipient, diluent or adjuvant for non-systemic administration to a subject in need thereof.
